(19) United States
(12) Patent Application Publication
(10) Pub. No.: US 2009/0286731 A1
(43) Pub. Date: Nov. 19, 2009

(54) METHODS AND COMPOSITIONS FOR THE TREATMENT OF XEROSTOMIA

(75) Inventors: Steven M. Anderson, Aurora, CO (US); Kirsten H. Limesand, Tucson, AZ (US)

Correspondence Address: FULBRIGHT & JAWORSKI L.L.P., 600 CONGRESS AVE., SUITE 2400, AUSTIN, TX, 78701, US

(73) Assignee: THE REGENTS OF THE UNIVERSITY OF COLORADO, A BODY CORPORATE, Boulder, CO (US)

(21) Appl. No.: 12/304,359

(22) PCT Filed: Jun. 12, 2007
(86) PCT No.: PCT/US2007/070924

§ 371 Date: Jul. 28, 2009

Related U.S. Application Data
(60) Provisional application No. 60/812,792, filed on Jun. 12, 2006.
(60) Provisional application No. 60/830,236, filed on Jul. 12, 2006.

(51) Int. Cl. A61K 38/18; A61K 38/16

(52) U.S. Cl. 514/12

(57) ABSTRACT

Methods for the treatment of xerostomia are described, hi particular, the present invention takes advantage of the inventors' observation that xerostomia is caused by induction of apoptosis, and can be inhibited by interfering with the cellular processes that trigger apoptosis in cells receiving chemo- and/or radiotherapy.

The present application claims benefit of priority to U.S. Provisional Application Nos. 60/812,792 filed Jun. 12, 2006 and 60/830,236 filed Jul. 12, 2006, the entire contents of each of these applications being incorporated by reference herein.

The government owns rights in this application pursuant to funding from the National Institutes of Health under grant nos. PO DE 12798 and F32 DE 14315.

### BACKGROUND OF THE INVENTION

A. Field of the Invention

The present invention is related generally to the field of oncology and cancer treatment. In particular, the invention relates to compositions and methods of preventing xerostomia.

B. Related Art

Patients with head and neck cancer are afflicted with a disease that often has profound effects on speech, swallowing and cosmesis. Most head and neck cancers begin in the cells that line the mucosal surfaces in the head and neck area, e.g., mouth, nose, and throat. Mucosal surfaces are moist tissues lining hollow organs and cavities of the body open to the environment. Normal mucosal cells look like scales (squamous) under the microscope, so head and neck cancers are often referred to as squamous cell carcinomas. Some head and neck cancers begin in other cells, such as glandular cells, which are called adenocarciomas.

The treatment plan for an individual patient depends on a number of factors, including the exact location of the tumor, the stage of the cancer, and the person's age and general health. For example, a surgeon may remove the cancer and some of the healthy tissue around it. Lymph nodes in the neck may also be removed, if the doctor suspects that the cancer has spread. Another common therapy is radiation, which involves the use of high-energy x-rays to kill cancer cells. Radiation may come from a machine outside the body, called external beam radiotherapy, but can also come from radioactive materials placed directly into or near the tumor. Chemotherapy, which involves the administration of cancer killing drugs, is another therapy of choice. Surgery, radiation and/or chemotherapy can be used in combination as well.

Unfortunately, chemo- and radiotherapy can cause unwanted destruction of salivary glands resulting in a condition know as xerostomia, or “dry mouth.” Some 40,000 patients are affected by xerostomia in the U.S. each year, and suffer from reduced quality of life due to difficulties in speech and swallowing, as well as an increased susceptibility to infection. The literature indicates that irradiation-induced salivary hypofunction is due to necrosis of the salivary glands. To date, the most common approach to preventing xerostomia is to reduce the total maximal exposure of radiation, better radiation targeting, and glandular shielding. However, improved methods of preventing xerostomia are needed.

### SUMMARY OF THE INVENTION

Thus, in accordance with the present invention, there is provided a method of reducing chemotherapy- or radiation-induced xerostomia in a subject having head & neck cancer comprising administering to said subject an inhibitor of apoptosis. The inhibitor may be administered local to cancer, such as by injection into a salivary duct or oral lavage, or administered systemically. The inhibitor may be an activator of the Akt pathway, such as IGF-1, or may be an inhibitor of the p53 pathway, such as activator of mdm2. The inhibitor may be administered to said subject prior to receiving radiation or chemotherapy, concurrent with receiving radiation or chemotherapy, or prior to and concurrent with receiving radiation or chemotherapy. The inhibitor may be administered to said subject with 1 hour of receiving radiation or chemotherapy, and may be administered more than once. The method may further comprise administering chemotherapy or radiation therapy, such as x-radiation, γ-radiation, or microwave radiation, or cisplatin, doxorubicin, taxol, etoposide, verapamil, podophyllotoxin, 5-fluorouracil (5-FU), cetuximab, docetaxel, carboplatin, and paclitaxel. The head & neck cancer may be squamous cell carcinoma or adenocarcinoma.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” “About” means plus or minus 5% of the stated value.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-D—Identification of myr-Akt1 transgenic mice. Tissue lysates were prepared from four week old female mice. In FIG. 1A, 100 μg of protein lysates isolated from the salivary glands of mice in each founder line were electrophoresed on an 8% SDS-PAGE gel, and immunoblotted with an antibody that detects the HA tag. Membranes were stripped and immunoblotted with a total ERK antibody to confirm equal loading of lanes (bottom panel). In FIG. 1B, total Akt levels were analyzed in FVB salivary glands and compared to salivary glands from myr-Akt1 founder line 1699. Membranes were stripped and immunoblotted with a total ERK antibody to confirm equal loading of lanes (bottom panel). In FIG. 1C, total Akt was immunoprecipitated from tissue lysates (300 μg; N=4 for each genotype) and used in an immune complex protein kinase assay containing ³²P [γ-³²P]ATP and a specific substrate peptide (RPRAATF). The incorporation of ³²P into the substrate peptide was determined by liquid scintillation counting and was plotted as average counts per minute (CPM). In FIG. 1D, phosphorylation of GSK3α/β on serine⁹ was analyzed in tissue lysates by immunoblot analysis with anti-phospho-GSK3. Membranes were stripped and immunoblotted with a total GSK3β antibody to confirm equal loading of lanes (bottom panel).

FIGS. 2A-F—Histological examination of salivary gland structure (200×). Submandibular (FIGS. 2A,D), sublingual (FIGS. 2B,E) and parotid (FIGS. 2C,F) salivary glands from four week old female animals were isolated from myr-Akt1 transgenic (FIGS. 2A-C) and FVB control (FIGS. 2D-F) mice. Tissues were fixed in 10% formalin, sectioned, stained with hematoxylin and eosin, and examined by bright field microscopy. Magnification bars represent 100 μm.

FIGS. 3A-B—Induction of apoptosis by etoposide is diminished in salivary acinar cells from myr-Akt1 transgenic mice. Four days post-initiation of the cultures, primary salivary acinar cells were treated with increasing doses of etoposide for eighteen hours. In FIG. 3A, all adherent and nonadherent primary parotid acinar cells were collected and lysed in caspase lysis buffer (BioMol QuantiZyme Colormetric Assay kit) and 15 μg of cell lysate was used to analyze the level of enzyme activity for each sample in quadruplicate. The ability of the enzyme to cleave a chromogenic substrate was read at A₄₀₅ every ten minutes for seven hours and the -fold increase relative to untreated parotid cells is plotted. No statistical differences were detected between untreated FVB and untreated myr-Akt1 parotid lysates (P>0.62). In FIG. 3B, primary submandibular acinar cells were collected in caspase lysis buffer as described above and the -fold increase relative to untreated submandibular cells is plotted. No statistical differences were detected between untreated FVB and untreated myr-Akt1 submandibular lysates (P>0.95). Graphs of caspase activity represent all data points collected from five independent experiments and Student's t-test p values were calculated using Microsoft Excel comparing two different treatment groups. Asterisks designate significant differences (P≦0.05) between myr-Akt1 and FVB.

FIGS. 4A-B—Salivary acinar cells from myr-Akt1 transgenic mice are resistant to γ-irradiation-induced apoptosis. Four days post-initiation of the cultures, primary salivary acinar cells were treated with increasing doses of γ-irradiation and the extent of apoptosis quantitated after 24 hours. In FIG. 4A, all adherent and nonadherent primary parotid acinar cells were collected, the activation of caspase-3 determined as described in FIG. 4A, and the fold increase relative to untreated parotid cells is plotted. In FIG. 4B, primary submandibular acinar cells exposed to varying concentrations of γ-irradiation and the activation of caspase-3 quantitated as described in FIG. 4B and the fold increase relative to untreated submandibular cells plotted. Caspase graphs represent all data points collected from five independent experiments (Mean±SEM) and Student's t-test p values were calculated using Microsoft Excel comparing two different treatment groups. Asterisks designate significant differences (P≦0.05) between myr-Akt1 and FVB using a two sample t-test.

FIGS. 5A-C—Reduced apoptosis in myr-Akt1 transgenic mice following targeted head and neck γ-irradiation in vivo. In FIG. 5A, four-week old female FVB (left panel) and myr-Akt1 transgenic (right panel) mice were exposed to 5Gy irradiation and the number of apoptotic cells in the parotid glands was quantitated by anti-activated caspase-3 immunohistochemistry 24 hrs post-irradiation. Magnification bars represent 100 microns. In FIG. 5B, at 8 and 24 hrs after irradiation, the percent of activated caspase-3 positive cells was calculated from parotid glands isolated from FVB and myr-Akt1 transgenic mice. Graph represents all data points from three mice per group (mean±SEM). Cell counts were performed on a minimum of five fields of view per slide from three mice (total cells counted ranged from 1800-2500 per mouse). Asterisks designate significant differences (P≦0.05) between myr-Akt1 and FVB using a two sample t-test in Microsoft Excel. In FIG. 5C, at 8 and 24 hrs after irradiation, tissue lysates were prepared as described for FIG. 1 and immunoblotted with anti-phospho-p53 (serine 8, top panel) or anti-HA to detect transgene (second panel). Membranes were stripped and immunoblotted with an anti-ERK to confirm equal loading of lanes (bottom panel).

FIGS. 6A-D—Expression of p53 family members and p21^{WAF1} is reduced in salivary glands of myr-Akt1 transgenic mice. In FIG. 6A, tissue lysates were immunoblotted with anti-p53 (DO12) to detect total levels of p53 (top panel) or levels of phosphorylated MDM2^{serine163} (second panel) or total levels of MDM2 (third panel). Membranes were stripped and immunoblotted with an anti-ERK to confirm equal loading of lanes (bottom panel). In FIG. 6B, total cellular RNA isolated from untreated parotid glands (N=4/genotype) was analyzed by real-time RT/PCR for basal concentrations of p21^{WAF1}. p21^{WAF1} Ct values were normalized by S15 Ct values and are expressed as a ratio of p21^{WAF1} expression/S15 expression. Student's t-test p value was calculated using Microsoft Excel comparing two different treatment groups (P≦0.025). In FIG. 6C, parotid tissue lysates were analyzed for total levels of p21^{WAF1}. Membrane was stripped and immunoblotted with an anti-ERK to confirm equal loading of lanes (bottom panel). In FIG. 6D, tissue lysates were analyzed for total levels of p63 (top panel) and p73 (middle panel). Membranes were stripped and immunoblotted with an anti-ERK to confirm equal loading of lanes (bottom panel). Immunoblots are representative of three individual mice.

FIGS. 7A-B—Diminished levels of p53 in primary myr-Akt1 salivary acinar cells following DNA damage. In FIG. 7A, cell lysates were collected as described in the materials and methods and immunoblotted with an anti-phospho-p53 (serine¹⁸, top panel) or anti-p53 (DO12, middle panel) eighteen hours after exposure to different concentrations of etoposide. Membranes were also stripped and immunoblotted with a β-tubulin antibody to confirm equal loading of lanes (bottom panel). In FIG. 7B, cell lysates were collected 24 hrs after exposure to varying doses of γ-irradiation, lysates were collected, and immunoblotted with an anti-phospho-p53 (serine¹⁸, top panel) or anti-p53 (DO12, middle panel). Membranes were also stripped and immunoblotted with a β-tubulin antibody to confirm equal loading of lanes (bottom panel). Results shown are representative of three independent experiments.

FIGS. 8A-B—Diminished expression of p21^{WAF1} following DNA damage in myr-Akt1 salivary acinar cells. Total RNA was isolated from salivary acinar cells as described in the materials and methods. In FIG. 8A, RNA isolated from primary FVB or myr-Akt1 parotid acinar cells 18 hrs after treatment with etoposide and the amount of p21^{WAF1} RNA was determined by qRT/PCR. In FIG. 8B, total cellular RNA was extracted from primary FVB or myr-Akt1 submandibular acinar cells 12 hrs following γ-irradiation treatment and analyzed for p21^{WAF1} expression by qRT/PCR. p21^{WAF1} Ct values were normalized by the ribosomal protein S15 Ct values and are expressed as a ratio of p21^{WAF1} expression/S15 expression. Asterisks designate significant differences (P≦0.05) between myr-Akt1 and FVB using a two sample t-test.

FIGS. 9A-C—MDM2 expression is required for myr-Akt1 mediated suppression of apoptosis in salivary acinar cells. In FIG. 9A, primary salivary acinar cells isolated from myr-Akt1 mice were transfected with various control or MDM2 specific siRNA molecules. Cell lysates were collected 24-72 hrs after transfection and immunoblotted with an anti-MDM2 antibody (top panel) or anti-p53 (DO12, middle panel). Membranes were also stripped and immunoblotted with a β-tubulin antibody to confirm equal loading of lanes (bottom panel). In FIG. 9B, primary salivary acinar cells isolated from myr-Akt1 mice were transfected with siRNA molecules as in FIG. 9A for 30 hrs and then treated with 150 μM etoposide for 18 hrs. Activation of caspase-3 was determined as described in the materials and methods, and plotted as the fold-increase relative to untreated parotid cells. The last lane depicts the induction of caspase-3 activity in FVB primary cells following treatment with 150 μM etoposide for 18 hrs. Data was collected from four independent experiments (mean±SEM) and Student's t-test p values were calculated using Microsoft Excel comparing two different treatment groups. Asterisks designate significant differences (P≦0.05) between myr-Akt1 etoposide and individual siRNA molecules using a two sample t-test. In FIG. 9C, primary salivary acinar cells isolated from myr-Akt1 mice were transfected with various control or MDM2 specific siRNA molecules for 30 hrs followed by 150 μM etoposide for 18 hrs similar to B. Cell lysates were collected and immunoblotted with a phosphorylation specific anti-Chk1 antibody (serine³⁴⁵, top panel), phosphorylation specific anti-p53 (serine¹⁸, third panel) or anti-total p53 (bottom panel). Membranes were also stripped and immunoblotted with a total Chk1 antibody to confirm equal loading of lanes (second panel). In lanes 6 and 7, primary salivary acinar cells isolated from myr-Akt1 mice were transfected with siRNA molecules targeting MDM2 for 48 hrs.

FIGS. 10A-B—Reduced apoptosis in myr-Akt1 parotid glands following exposure to ionizing radiation. (FIG. 10A) Four-week old female FVB and myr-Akt1 mice were exposed to 0.5, 1, or 5Gy γ-irradiation and parotid salivary glands were removed 24 hrs post-irradiation. Tissues were embedded into paraffin and sections were stained for activated caspase-3. The number of caspase-3 positive cells is graphed as a percentage of the total number of cells per field of view. (FIG. 10B) Four-week old female FVB and myr-Akt1 mice were exposed to 1Gy γ-irradiation and parotid salivary glands were removed at 24 and 48 hrs. Tissues were processed for activated caspase-3 immunohistochemistry as described in FIG. 10A. Five fields of view were quantitated for each tissue section.

FIG. 11—Rescue of salivary flow rates 3 days after single γ-irradiation exposure in Myr-Akt1 mice. Four-week old female FVB and myr-Akt1 mice were exposed to 1, 2, or 5Gy γ-irradiation. Three days after irradiation total saliva was collected (over a five-minute period) following carbachol injection. Statistical analysis was performed using Student's t-test in Microsoft Excel.

FIGS. 12A-C—IGF1 activation of Akt in vivo and rescue of salivary flow rates 3 days after single γ-irradiation exposure. (FIG. 12A) FVB mice received an injection of 1, 5, 10, or 50 ng/ml recombinant IGF1. Tissue lysates were collected for immunoblotting five minutes after injection and membranes were probed for activation of Akt using a phosphorylation specific antibody. (FIG. 12B) FVB mice received an injection of 5 μg/ml recombinant IGF1 and tissue lysates were collected for immunoblotting 0, 5, 10 or 30 min after injection. Membranes were probed for activated Akt as described in FIG. 12A. Membranes were stripped and re-probed with a total antibody against ERK1/2 as a loading control in both A and B. (FIG. 12C) Four-week old female FVB mice were injected with 5 μg/ml recombinant IGF1 immediately prior to treatment with 1Gy γ-irradiation. Total saliva was collected following carbachol injection as described in FIG. 11 three days after radiation exposure. For Untreated, N=10; for IGF1, N=5; for 1 Gy, N=10, and for 1 Gy+IGF1, N=7. Statistical analysis was performed using Student's t-test in Microsoft Excel.

FIG. 13—Rescue of salivary flow rates 30 days after single γ-irradiation exposure by Myr-Akt1 or injection with IGF1. Four-week old female FVB, myr-Akt1 and FVB mice injected with 5 μg/ml recombinant IGF1 were exposed to 1Gy γ-irradiation. IGF1 injections were performed immediately prior to radiation exposure as described in FIGS. 12A-C. Thirty days after exposure to γ-irradiation total saliva was collected following carbachol injection as described in FIG. 11. For unirradiated, untreated, N=8; for unirradiated myr-Akt1, N=8; for unirradiated IGF1 injected, N=8; for 1 Gy irradiated FVB, N=8, for myr-Akt1 riiadiated with 1 Gy, N=6, for FVB that received IGF1 and I Gy, N=6. Statistical analysis was performed using multiple comparison testing in the SAS system.

### DETAILED DESCRIPTION OF THE PREFERRED EMBODIMENTS

It is well known that exposure of the head and neck region of humans or animals to chemotherapy or radiation causes salivary gland hypofunction, also referred to as xerostomia. It has been widely held that the tissue damage induced by radiation of the salivary gland was caused by necrosis. The inventors' research led them to believe that this view was incorrect, and that in fact, radiation of the head and neck caused apoptosis in the salivary cells, often within 8-24 hours. This hypothesis, if confirmed, could provide a paradigm shift in the possible treatment of xerostomia, namely, prevention of apoptosis in salivary acinar cells following radiation and chemotherapy.

The inventors' studies found that etoposide and γ-irradiation induce apoptosis of salivary acinar cells from FVB control mice in vitro and in vivo; however, apoptosis was reduced in transgenic mice expressing a constitutively activated mutant of Akt1 (myr-Akt1). Expression of myr-Akt1 in the salivary glands results in a significant reduction in phosphorylation of p53 at serine[¹⁸], total p53 protein accumulation, and p21^{WAF1} mRNA following etoposide or γ-irradiation of primary salivary acinar cells. The reduced level of p53 protein in myr-Akt1 salivary glands in vivo corresponds with an increase in MDM2 phosphorylation, suggesting the Akt/MDM2/p53 pathway is responsible for suppression of apoptosis. Reduction of MDM2 levels in myr-Akt1 primary salivary acinar cells using siRNA increases levels of p53 protein and renders these cells susceptible to etoposide-induced apoptosis in spite of the presence of activated Akt1. These results indicate that MDM2 is a critical substrate of activated Akt1 in the suppression of p53-dependent apoptosis in vivo. They also indicate that prevention of apoptosis in salivary cells will be protective from xerostomia.

### I. HEAD & NECK CANCER

Head and neck cancers account for approximately 3 to 5 percent of all cancers in the United States. These cancers are more common in men and in people over age 50. It is estimated that about 39,000 men and women in this country will develop head and neck cancer in 2005. Tobacco and alcohol use are the most important risk factors for head and neck cancers, particularly those of the oral cavity, oropharynx, hypopharynx, and larynx. Eighty-five percent of head and neck cancers are linked to tobacco use. People who use both tobacco and alcohol are at greater risk for developing these cancers than people who use either tobacco or alcohol alone.

Region specific risk factors include: oral cavity—sun exposure (lip); possibly human papillomavirus (HPV) infection; salivary glands—radiation to the head and neck. This exposure can come from diagnostic x-rays or from radiation therapy for noncancerous conditions or cancer; paranasal sinuses and nasal cavity—certain industrial exposures, such as wood or nickel dust inhalation (tobacco and alcohol use may play less of a role in this type of cancer); nasopharynx—Asian, particularly Chinese, ancestry; Epstein-Barr virus infection; occupational exposure to wood dust; and consumption of certain preservatives or salted foods; oropharynx—poor oral hygiene; HPV infection and the use of mouthwash that has a high alcohol content are possible, but not proven, risk factors; hypopharynx—Plummer-Vinson (also called Paterson-Kelly) syndrome, a rare disorder that results from iron and other nutritional deficiencies (this syndrome is characterized by severe anemia and leads to difficulty swallowing due to webs of tissue that grow across the upper part of the esophagus); larynx—exposure to airborne particles of asbestos, especially in the workplace. Immigrants from Southeast Asia who use paan (betel quid) in the mouth should be aware that this habit has been strongly associated with an increased risk for oral cancer. Also, consumption of mate, a tea-like beverage habitually consumed by South Americans, has been associated with an increased risk of cancers of the mouth, throat, esophagus, and larynx.

Most head and neck cancers begin in the cells that line the mucosal surfaces in the head and neck area, e.g., mouth, nose, and throat. Mucosal surfaces are moist tissues lining hollow organs and cavities of the body open to the environment. Normal mucosal cells look like scales (squamous) under the microscope, so head and neck cancers are often referred to as squamous cell carcinomas. Some head and neck cancers begin in other types of cells. For example, cancers that begin in glandular cells are called adenocarcinomas. Symptoms of several head and neck cancer sites include a lump or sore that does not heal, a sore throat that does not go away, difficulty swallowing, and a change or hoarseness in the voice. Other symptoms may include the following: oral cavity—a white or red patch on the gums, tongue, or lining of the mouth; a swelling of the jaw that causes dentures to fit poorly or become uncomfortable; and unusual bleeding or pain in the mouth; nasal cavity and sinuses—sinuses that are blocked and do not clear, chronic sinus infections that do not respond to treatment with antibiotics, bleeding through the nose, frequent headaches, swelling or other trouble with the eyes, pain in the upper teeth, or problems with dentures; salivary glands—swelling under the chin or around the jawbone; numbness or paralysis of the muscles in the face; or pain that does not go away in the face, chin, or neck; oropharynx and hypopharynx—ear pain; nasopharynx—trouble breathing or speaking, frequent headaches, pain or ringing in the ears, or trouble hearing; larynx—pain when swallowing, or ear pain; metastatic squamous neck cancer—pain in the neck or throat that does not go away.

To find the cause of symptoms, a doctor evaluates a person's medical history, performs a physical examination, and orders diagnostic tests. The exams and tests conducted may vary depending on the symptoms. Examination of a sample of tissue under the microscope is always necessary to confirm a diagnosis of cancer. Some exams and tests that may be useful are: physical examination may include visual inspection of the oral and nasal cavities, neck, throat, and tongue using a small mirror and/or lights, and the doctor may also feel for lumps on the neck, lips, gums, and cheeks; endoscopy is the use of a thin, lighted tube called an endoscope to examine areas inside the body, with the type of endoscope the doctor uses depending on the area being examined; laboratory tests examine samples of blood, urine, or other substances from the body; X-rays create images of areas inside the head and neck on film; CT (or CAT) scan is a series of detailed pictures of areas inside the head and neck created by a computer linked to an x-ray machine; magnetic resonance imaging (or MRI) uses a powerful magnet linked to a computer to create detailed pictures of areas inside the head and neck; PET scan uses sugar that is modified in a specific way so it is absorbed by cancer calls and appears as dark areas on the scan.; biopsy is the removal of tissue. A pathologist studies the tissue under a microscope to make a diagnosis. A biopsy is the only sure way to tell whether a person has cancer. If the diagnosis is cancer, the doctor will want to learn the stage (or extent) of disease. Staging is a careful attempt to find out whether the cancer has spread and, if so, to which parts of the body. Staging may involve an examination under anesthesia (in the operating room), x-rays and other imaging procedures, and laboratory tests. Knowing the stage of the disease helps the doctor plan treatment.

### II. XEROSTOMIA

Xerostomia is not a disease per se, but rather is a collection of symptoms that can be caused by a number of underlying diseases or environmental factors. Xerostomia can be caused by a large number of prescription and non-prescription drugs, including antihypertensives, antidepressants, analgesics, tranquilizers, diuretics and antihistamines, by cancer therapies such as chemotherapeutic drugs and radiation treatment; Sjogren's syndrome, an autoimmune disease; and other conditions such as bone marrow transplants, endocrine disorders, stress, anxiety, depression, and nutritional deficiencies. Nerve damage resulting from trauma to the head and neck area (e.g., surgery or wounds) may also be a cause of xerostomia.

Traditional treatment generally depends on the cause of the condition, and if possible, the inducing factor or factors should be eliminated or reduced. However, in many situations it will be difficult or impossible to eliminate the cause, such as when cancer therapy is required. Palliative treatments include pilocarpine (Salagen) 5 mg, qid; special food preparation—blended and moist foods are easier to swallow; artificial saliva (available over-the-counter); sipping plain water is usually preferred over artificial saliva by most patients; Biotene brand, over-the-counter, dry mouth products (toothpaste, alcohol-free mouth rinse and Oralbalance lubricating gel); avoidance of alcohol-based mouth rinses; and use of water and glycerin mixed in a small aerosol spray bottle.

### III. INHIBITION OF APOPTOSIS

The serine/threonine protein kinase Akt, also known as protein kinase B (PKB)¹, provides an important survival signal in many different tissues (Coffer et al., 1998; Datta et al., 1999; Kandel and Hay, 1999). Activation of Akt occurs in a PI3 kinase-dependent manner following stimulation of cells with a variety of growth factors that induce cell proliferation and cell survival (Datta et al., 1996). Potential substrates for Akt include procaspase-9 (Cardone et al., 1998), the pro-apoptotic Bcl-2 family member BAD (del Peso et al., 1997; Datta et al., 1997; Datta et al., 2000), and members of the Forkhead family of transcription factors (Brunet et al., 1999; del Peso et al., 1999). Other recently identified substrates for Akt include tuberous sclerosis complex (Tsc2), a regulator of the mammalian target of rapamycin (mTOR) (Inoki et al., 2002; Potter et al., 2002), Myt1, a regulator of G2/M phase transition (Okumura et al., 2002), Yes associated protein (YAP), a transcriptional coactivator of the p53 homologue p73 (Basu et al., 2003), and Chk1, a protein kinase activated by DNA damage whose activation is inhibited when phosphorylated by Akt (King et al., 2004). Expression of activated Akt1 in the mammary glands of transgenic mice significantly delays involution of mammary epithelial cells, a process that requires apoptosis (Hutchinson et al., 2001; Schwertfeger et al., 2001). Other constitutively activated Akt transgenic mouse systems have confirmed other signaling molecules that lie downstream of Akt including cyclin D, eIF4E, and mTOR (Hutchinson et al., 2001; Shioi et al., 2002; Wendel et al., 2004). It is well known that activated Akt suppresses apoptosis, although the in vivo mechanisms of this suppression are largely unknown. Akt can phosphorylate a number of substrates in vitro; however, the critical substrate(s) in vivo may be cell type and stimulus specific.

Upon DNA damage, p53 undergoes several post-translational modifications, including phosphorylation and acetylation which increases the stability of p53 dramatically (Inoki et al., 2002; Li et al., 2003; Meek, 1999; Michael and Oren, 2003; Woods and Vousden, 2001). The ATM (ataxia telangiectasia mutated) protein kinase senses double-stranded breaks or changes in the chromatin structure of DNA (Bakkenist and Kastan, 2003) and forms a complex with p53 leading to the phosphorylation p53 on serine¹⁵ (Meek, 1999). In addition to ATM, the related ATM-Rad3-related protein kinase (ATR), also phosphorylates p53 on serine¹⁵, suggesting that p53 modification and accumulation may be controlled by multiple kinases that are activated by different types of DNA damage (Bao et al., 2001; Tibbetts et al., 1999). ATM and ATR are both able to phosphorylate and activate Chk1 which leads to phosphorylation and destruction of CDC25A, and cell cycle arrest (Bartek et al. 2004); phosphorylation of Chk1 by Akt inhibits Chk1 and prevents cell cycle arrest (King et al., 2004). DNA damage induced activation of p53 causes cell cycle arrest, predominantly at the G₁-S transition, and activates DNA repair; however, if DNA damage is severe and repair is not effective, p53 also can induce apoptosis (Verheij and Bartelink, 2000).

Recently, Akt has been shown to modulate the activity of p53 through its substrate Murine Double Minute Clone 2 (MDM2) (Ogawara et al., 2002; Oren et al., 2002; Zhou et al., 2001). MDM2 is an E3 ubiquitin ligase that negatively regulates p53 transcriptional activity (Yin et al., 2002). Phosphorylation of MDM2 on serine¹⁶⁶ and serine¹⁸⁶ by Akt stimulates translocation of MDM2 to the nucleus where it binds to p53 and targets p53 degradation by the proteosome (Ogawara et al., 2002; Oren et al., 2002; Zhou et al., 2001). The correlation between Akt activation and MDM2 localization has also been extended in vivo to breast tumor tissues (Zhou et al., 2001). To date, analysis of the Akt/MDM2/p53 pathway has been largely confined to studies utilizing transient transfection of tissue culture cells; here we report that expression of MDM2 in primary cells that express activated Akt1 is critically important for the ability of Akt to suppress apoptosis following DNA damage. Thus, the present inventors suggest use of agonists of the Akt pathway including IGF1.

### IV. PHARMACEUTICAL COMPOSITIONS AND ROUTES OF ADMINISTRATION

Where clinical application of an inhibitor of apoptosis according to the present invention is contemplated, it will be necessary to prepare the complex as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Aqueous compositions of the present invention comprise an effective amount of the inhibitor of apoptosis, dissolved or dispersed in a pharmaceutically acceptable carrier or aqueous medium. Such compositions also are referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The inhibitor of apoptosis of the present invention may include classic pharmaceutical preparations. Administration of therapeutic compositions according to the present invention will be via any common route so long as the target tissue is available via that route. Administration may be by intradermal, subcutaneous, intramuscular, intraperitoneal, intraarterial or intravenous injection. In particular, local (intratumoral, intratumoral vasculature), regional to a tumor (salivary ducts, oral lavage), and systemic are contemplated. Such compositions would normally be administered as pharmaceutically acceptable compositions that include physiologically acceptable carriers, buffers or other excipients.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve, liquid or spray.

An effective amount of the therapeutic agent is determined based on the intended goal, i.e., for inhibition of apoptosis in salivary cells. The term “unit dose” refers to physically discrete units suitable for use in a subject, each unit containing a predetermined-quantity of the prophylactic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the composition also depend on the judgment of the practitioner and are peculiar to each individual.

### V. COMBINATION WITH THERAPY PROTOCOLS

The present invention contemplates, in addition to administering the inhibitor of apoptosis in combination with a chemotherapy or radiotherapy. Methods of treating head & neck cancers with drugs and/or radiation are well known to those of skill in the art, and need not be reiterated in detail here. Such methods may also be used prior or subsequent to surgery. In some cases, treatment of a tumor with a chemo- or radiotherapy may result in an inoperable tumor being rendered operable. Thus, therapeutic compositions may be provided in combination with the inhibitor of apoptosis. This process may involve administration to a patient of both agents at the same time. Alternatively, the inhibitor of apoptosis may precede or follow the chemo- or radiotherapy by intervals ranging from minutes to weeks. In embodiments where the two agents are applied at different times, one would generally ensure that a significant period of time did not expire between the time of each delivery, such that the inhibitor of apoptosis could still be able to exert a protective effect on the salivary cells of the patient. In such instances, it is contemplated that one would contact the cell with both agents within about 1 hour, 2 hours, 3 hours, 4 hours, 5 hours, 6 hours, 12 hours to about 24-72 hours of each other.

It also is conceivable that more than one administration of either the inhibitor of apoptosis and chemo- or radiotherapy will be desired. Various combinations may be employed, where inhibitor of apoptosis is “A” and the chemo- or radiotherapy is “B”:

| | | | | | |
|---|---|---|---|---|---|
| A/B/A | B/A/B | B/B/A | A/A/B | B/B/B/A | B/B/A/B |
| A/A/B/B | A/B/A/B | A/B/B/A | B/B/A/A | B/A/B/A | B/A/A/B |
| A/A/A/B | B/A/A/A | A/B/A/A | A/A/B/A | A/B/B/B | B/A/B/B |

A. Chemotherapies

Various drugs may be administered to a head & neck cancer patient. For example, agents that directly cross-link polynucleotides, specifically DNA, are envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, taxol, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors and subunits also lead to DNA damage. As such a number of polynucleotide precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Cetuximab and Docetaxel have been used successfully in combination therapies with radiation. Cetuximab (Ertibux®) is a monoclonal antibody directed to the EGF receptor inside the cell which prevents the receptor from being activated, in turn stopping the cancer cell from dividing. Docetaxel (Taxotere®) is an antineoplastic drug that interferes with the growth of cancer cells and slows their growth and spread in the body. Other combination drugs for use with radiation are cisplatin (Platinol®), fluorouracil (5-FU, Adrucil®), carboplatin (Paraplatin®), and paclitaxel (Taxol®).

The skilled artisan is directed to “Remington's Pharmaceutical Sciences,” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. Radiation

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of DNA damage, or the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

C. Surgery

A surgeon may choose to remove a tumor, and optionally and some of the healthy tissue around it (tumor margin). Lymph nodes in the neck may also be removed if the doctor suspects that the cancer has spread. Surgery may be followed by radiation treatment and/or chemotherapy. Head and neck surgery often changes the patient's ability to chew, swallow, or talk. The patient may look different after surgery, and the face and neck may be swollen. However, lymph node dissection can slow the flow of lymph, which may collect in the tissues; this swelling may last for longer periods. After a laryngectomy (surgery to remove the larynx), parts of the neck and throat may feel numb because nerves have been cut. If lymph nodes in the neck were removed, the shoulder and neck may be weak and stiff. Obviously, the location of the tumor will have a great impact on the ability of the surgeon to intervene and to fully remove the tumor, as well as on the patient's quality of life going forward. Tumors may be located as follows: oral cavity; nasal cavity and sinuses; salivary glands; oropharynx and hypopharynx; and larynx.

### VI. KITS

All the essential materials and reagents required for inhibiting xerostomia may be assembled together in a kit. This generally will comprise one or more selected inhibitors of apoptosis. Such kits will comprise distinct containers for each individual reagent. When the components of the kit are provided in one or more liquid solutions, the liquid solution preferably is an aqueous solution, with a sterile aqueous solution being particularly preferred. For in vivo use, the agent may be formulated into a pharmaceutically acceptable syringeable composition. In this case, the container means may itself be an inhalent, syringe, pipette, or other such like apparatus, from which the formulation may be applied to an affected area of the body, or even applied to and mixed with the other components of the kit. The components of the kit may also be provided in dried or lyophilized forms. When reagents or components are provided as a dried form, reconstitution generally is by the addition of a suitable solvent. It is envisioned that the solvent also may be provided in another container means.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalent, syringe, pipette, forceps, measured spoon, eye dropper or any such medically approved delivery vehicle.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### A. Materials and Methods

Mice. Myr-Akt1 transgenic mice were generated using standard techniques as described previously (Schwertfeger et al., 2001) by the Transgenic Mouse Core of the University of Colorado Cancer Center. FVB mice were purchased from Taconic Laboratories (Germantown, N.Y.). Genomic tail DNA was extracted from the founder mice by Proteinase K digestion followed by phenol-chloroform extraction and ethanol precipitation (Schwertfeger et al., 2001). Transgenic mice were identified using PCR as previously described. The forward primer was from the sequence of Akt1 (5′-GCC GCT ACT ATG CCA TGA AGA-3′) (SEQ ID NO:1) and the reverse primer was targeted against the HA epitope (5′-GTA ATC TGG AAC ATC GTA TGG GTA-3′) (SEQ ID NO:2) (Schwertfeger et al., 2001). Animals were maintained in accordance with protocols approved by the University of Colorado Health Sciences Center Laboratory Animal Care and Use Committee.

Immunoblotting. Tissues were lysed in RIPA (150 mM NaCl, 50 mM Tris, pH 7.4, 2 mM EGTA, 1% Triton X-100, 0.25% sodium deoxycholate) supplemented with protease inhibitor cocktail (Sigma Chemical Company, St. Louis, Mo.), 100 μg/ml phenylmethylsulfonyl fluoride, and 100 U/ml aprotinin (Pierce Chemical Company, Rockford, Ill.). Tissues were then boiled for ten minutes, chilled on ice, and disrupted by sonication until homogeneous (Schwertfeger et al., 2001). Primary salivary acinar cells were lysed in JNK lysis buffer (25 mM HEPES, pH 7.5, 20 mM β-glycerophosphate, 0.1 mM sodium orthovandadate, 0.1% Triton X-100, 0.3M NaCl, 1.5 mM MgCl₂, 0.2 mM EDTA, 0.5 mM dithiothreitol, and 10 mM NaF) supplemented with aprotinin (4 μg/ml), Prefebloc (0.5 mg/ml), and leupeptin (2 μg/ml) (Pierce Chemical Company) and 1 mM sodium orthovandate, and the lysates clarified by centrifugation at 4° C. for 30 min at 13,000 rpm in a refrigerated Savant microcentrifuge (Anderson et al., 1999). Protein concentrations were determined using the BCA Protein Assay Kit (Pierce Chemical Company). For immunoblotting, 100 μg of whole cellular protein was resolved on an 8 or 10% polyacrylamide gel, transferred to Immunobilon membrane (Millipore Corporation, Bedford, Mass.), and immunoblotted. Anti-HA antibody was purchased from Roche Diagnostics (Indianapolis, Ind.). Anti-ERK antibody was purchased from Promega Corporation (Madison, Wis.), while anti-phosphorylated-p53 (serine¹⁵), anti-phosphorylated-GSK3α/β (serine⁹), anti-GSK3β and anti-phosphorylated-MDM2 (serine¹⁶⁶) were acquired from Cell Signaling Technologies (Beverly, Mass.). Serine¹⁵ in human p53 corresponds to serine¹⁸ in the mouse p53 and this antibody detects both species (Sluss et al., 2004). Serine¹⁶⁶ in human MDM2 corresponds to serine¹⁶³ in the mouse genome and this antibody also detects both species. Anti-p53 (DO-12) was obtained from Novacastra Laboratories (Burlingame, Calif.), anti-p73 (Ab-4) and anti-tubulin were purchased from Neomarkers (Fremont, Calif.), and anti-p63 and anti-MDM2 (Ab-4) were acquired from Oncogene Research Products (San Diego, Calif.). The inventors have also used Imgenex antibodies IMG-246 and IMG-259 for detection of p73 with similar results and these antibodies have been shown to detect endogenous p73 (Sayan et al., 2005). Anti-phosphorylated ATM, anti-Chk1, and anti-phosphoryalted Chk1 (serine³⁴⁵) were from Cell Signaling Technologies. Secondary antibodies were conjugated with horseradish peroxidase and the anti-HRP conjugated rabbit antibody was purchased from Bio-Rad (Hercules, Calif.) while the anti-HRP conjugated mouse antibody was obtained from Amersham (Arlington Heights, Ill.). Enhanced chemiluminescence lighting (Pierce Chemical Company) was used according to the manufacturer's instructions to detect immunoblotted proteins. In some cases, membranes were then stripped as previously described (Hamilton et al., 2001), reblocked in Tris-buffered saline with Tween-20 (TBST; 10 mM Tris pH 7.4, 150 mM NaCl and 0.05% Tween-20) with 5% nonfat dry milk (Carnation), and probed with a second antibody. The activation of Akt kinase activity was quantitated using a radioactive Akt kinase assay kit (Upstate Biotechnology, Lake Placid, N.Y.) using 300 μg of tissue lysates according to manufacturer's instructions (Limesand et al., 2003).

Histology. Tissues were fixed in 10% neutral buffered formalin, and then embedded in paraffin. Tissue sections were cut at 4 μm and processed for standard staining with hematoxylin and eosin by the Histology Service of the Department of Pathology at the University of Colorado School of Medicine. Tissue sections were observed by standard light microscopy and photomicrographs were taken with Nikon Eclipse E600 microscope with Spot Diagnostic imaging software.

DNA content. Genomic DNA was extracted from myr-Akt1 or FVB salivary glands by Proteinase K digestion followed by phenol-chloroform extraction and ethanol precipitation. Submandibular and parotid DNAs were diluted 1:1000 and sublingual DNA was diluted 1:10 prior to analysis. DNA concentrations were determined using a PicoGreen dsDNA Quantitation kit (Molecular Probes) and analyzed in triplicate. Sample fluorescence (excitation ˜480 nm, emission ˜520 nm) was measured on a microplate reader (Molecular Devices, Sunnyvale, Calif.). A lambda DNA standard provided in the kit was used to generate a standard curve.

Saliva collection. Mice were injected intraperitoneally with 0.25 mg carbachol per kg body weight. Saliva was collected immediately following injection for five min and chilled on ice. Total proteins present in the collected saliva were analyzed by resolving 25 μg total protein on a 10% SDS-PAGE gel and the gel stained with Gel Blue reagent (Pierce Chemical Company) according to manufacturer's instructions.

Immunofluorescent staining. Salivary glands to be examined for amylase staining were removed, fixed in 10% neutral buffered formalin overnight, and subsequently embedded in Tissue Freezing Medium (Fisher Scientific, Pittsburgh, Pa.). Sectioning (7 μm) and staining were performed by the Histology Core, Barbara Davis Center for Childhood Diabetes (Denver, Colo.) (Jensen et al., 1996). Photomicrographs were taken with Nikon Eclipse E600 microscope using FITC (amylase) and UV (Hoerst) filters and images were merged as described above.

Generation and cultivation of primary salivary acinar cells. Primary salivary acinar cells were prepared from 4-5 week old female FVB or myr-Akt1 transgenic mice. Mice were anesthetized with avertin (0.4-0.6 mg/gm, IP) and primary parotid or submandibular acinar cells were prepared under sterile conditions similar to previously published protocols (Limesand et al., 2003a; Limesand et al., 2003b; Quissell et al., 1997). A 1% vol/vol cell suspension was seeded onto collagen-coated dishes or coverslips (Falcon/Becton Dickenson, Fairlawn, N.J.) and cultures were approximately 80% confluent after five days in culture. Untreated cells were examined by light microscopy to ensure an enriched population of acinar cells. Photomicroscopy was accomplished using an Olympus CK2 inverted microscope and imaged on Kodak TMAX100 film. 5-Bromo-2′deoxy-uridine (BrdU) labeling was performed for 30 min using the Labeling and Detection Kit I (Roche Diagnostics) according to manufacturer's instructions and immunofluorescent nuclei were imaged on a Nikon Eclipse E600 microscope using an FITC filter. Cell counts were performed on a minimum of six fields of view per slide from three independent experiments (total cells counted ranged from 850-2500 per group).

Induction and quantitation of apoptosis. Primary mouse salivary acinar cells were treated with varying doses of etoposide (50-200 μM) or ionizing radiation (0.25-5Gy). Etoposide was purchased from Sigma Chemical Company. Exposure to ionizing radiation was performed using a cobalt source (Model: GB150, Type: B(U), Serial: 32R from Atomic Energy of Canada Ltd). Lysates were prepared in JNK lysis buffer, as described above, 18 hours after treatment with etoposide or 24 hrs after treatment with γ-irradiation (Anderson et al., 1999; Limesand et al., 2003b). Activation of caspase 3 was quantitated using BioMol QuantiZyme Colormetric Assay kit (Plymouth Meeting, Pa.). The adherent and floating cells were collected from a 100 mm² dish and lysed in caspase lysis buffer supplemented with 0.1% Triton-X, aprotinin (4 μg/ml), Prefebloc (0.5 mg/ml), and leupeptin (2 μg/ml) according to manufacturer's instructions and previously published reports (Anderson et al., 1999; Matassa et al., 2001). Caspase-3 activity in 15 μg of cellular lysate was measured by the cleavage of Ac-DEVD-pNA substrate and absorbance at A₄₀₅ was quantitated in a microtiter plate reader (Molecular Devices) at 10-minute intervals for 7 hrs. Cells for TdT-mediated dUTP nick end labeling (TUNEL) assay were fixed in 2% paraformaldehyde and detection of apoptotic cells was performed using the Cell Death kit (Roche Diagnostics). Cell counts were performed on a minimum of six fields of view per slide from three independent experiments.

For in vivo experiments, four-week old female FVB and myr-Akt1 transgenic mice were anesthetized with avertin (0.4-0.6 mg/gm, IP) and the head and neck region was exposed to ionizing radiation using a cobalt source (Model: GB150, Type: B(U), Serial: 32R from Atomic Energy of Canada Ltd). The rest of the body was shielded with ˜6 mm lead to avoid systemic effects of γ-irradiation. Animals were treated in accordance with protocols approved by the University of Colorado Health Sciences Center Laboratory Animal Care and Use Committee.

Salivary glands were removed 8 and 24 hrs post-irradiation, fixed in 10% neutral buffered formalin, and then embedded in paraffin. Slides were heated to 60° C. for 45 min, then rehydrated in citrosolve, graded alcohols and dH₂O washes. For antigen retrieval, slides were placed in citrate buffer in a pressure cooker, heated in a microwave for 20 min and allowed to cool for 20 min. After washes, slides were blocked with aviden and biotin. Anti-activated caspase-3 (Cell Signaling #9661) was layered on at 1:100 dilution in Biomedia primary antibody diluting medium and allowed to incubate overnight at 4° C. on a rotating plate. Non-specific peroxidase activity was quenched with 1% H₂O₂ followed by secondary antibody (goat anti-rabbit at 1:200 dilution) incubation at room temp for 50 min. Vector ABC was incubated for 30 min. at room temperature and color development was achieved with Biogenex DAB incubation for 1-2 min. Slides were counterstained with Gill's Hematoxylin, dehydrated, and coverslipped with Protexx. Cell counts were performed on a minimum of five fields of view per slide from three mice (total cells counted ranged from 1800-2500 per mouse).

RNA isolation and quantitative RT/PCR. The adherent and floating primary cells were collected from a 100 mm2 dish and lysed in RLT buffer (Qiagen, Valencia, Calif.). Cells were homogenized using QiaShredder columns (Qiagen) and frozen at −80° C. until isolation procedure. Cellular homogenates were thawed at 37° C. and RNA was isolated using the RNeasy Qiagen column procedure. For preparation of RNA from tissue, salivary glands were isolated from four-week old FVB or myr-Akt1 female mice, cleaned of connective tissue and stored in RNA later (Qiagen). Samples were lysed in RLT lysis buffer and homogenized using a polytron (Rudolph et al., 2003). Total RNA was treated with DNase 1 using the DNA-free kit according to manufacturer's instructions (Ambion, Austin, Tex.) or using the on-column digestion protocol (Qiagen). Quantitative RT-PCR was conducted in triplicate for each RNA sample according to DeRyckere and DeGregori (2002), using the DNA Engine Opticon 2™ System (MJ Research Inc., Waltham, Mass.). Briefly, 25 ng of total RNA was added to the RT-PCR reaction buffer containing 0.2 μM 5′ and 3′ primers, 305 mU/μl of anti-RNase (Ambion), 76 mU/μl of M-MLV reverse transcriptase (Invitrogen), and a 1×SYBR® Green MasterMix (Applied Biosystems, Warrington, UK) at a final volume of 25 μl. The 96 well microplate was capped and centrifuged at 3300×g for 4 min. RNA was reverse transcribed at 48° C. for 30 min and the reverse transcriptase inactivated by a 10 min incubation at 95° C. DNA amplification was performed with 40 cycles of 95° C. for 15 sec, 60° C. for 60 sec, and fluorescent detection at 72° C. A melting curve following DNA amplification was obtained by increasing the temperature from 72-99.9° C. with a fluorescent detection every 0.1° C./sec to ensure a single amplification product. Background fluorescence was determined between cycles 1 through 18 and was used to calculate the threshold value, 5 standard deviations greater than background. Differences in starting concentrations were determined by the cycle number (Ct) at which the fluorescent intensity crossed the threshold for each sample. The Ct values were subtracted for the total number of cycles (40) and normalized with ribosomal protein S15 RNA to produce a relative abundance of starting RNA concentrations (Kitagawa et al., 1991). Homogeneity of variation was tested with an F-test and difference determined with an unpaired student's T-test for equal sample variation. Primer sequences for S15 were forward 5′-ATC ATT CTG CCC GAG ATG GTG-3′ (SEQ ID NO:3) and reverse 5′-TGC TTT ACG GGC TTG TAG GTG-3′ (SEQ ID NO:4). Primer sequences for p21WAF1 were generated according to previously published protocols (Wan and DeGregori, 2003) (forward 5′-GCC ACA GCG ACC ATG TCC AA-3′ (SEQ ID NO:5) and reverse 5′-GCG TCT CCG TGA CGA AGT CAA A-3′) (SEQ ID NO:6) and all primers were synthesized by Invitrogen. Quantitation of p73 and p63 RNA levels by real-time RT/PCR used the following primers; p73 forward 5′-TCT TCC TCC TCC ACC TT-3′ (SEQ ID NO:7) and reverse 5′-TGC TGA GCA AAT TGA ACT GC-3′ (SEQ ID NO:8) (Wan and DeGregori, 2003); and p63 forward 5′-CAG CAC CAG CAC CTA CTT CA-3′ (SEQ ID NO:9) and reverse 5′-GAT AAG CTG GCT CAC GGA AG-3′ (SEQ ID NO:10). Validated primer sets for p53 and Bax quantitative RT/PCR were purchased from Qiagen (QuantiTect Primer Assays) and used with the QuantiTect SYBR Green RT/PCR reagents according to manufacturer's instructions.

siRNA transfection. Custom siRNA constructs were designed by Qiagen based on murine MDM2 (GenBank Accession number NM_{—}010786) or purchased from Ambion (Catalog #67963 and 68152). Four days after initiation of primary salivary acinar cultures, 5 μg of MDM2 siRNA was complexed with RNAiFect (Qiagen) at a ratio of 1:9 according to manufacturer's instructions. Twenty-four to seventy-two hours after transfection, MDM2 protein expression in primary myr-Akt1or FVB salivary acinar cells was determined by immunoblot as described above. For analysis of apoptosis, primary myr-Akt1 salivary acinar cells were transfected with MDM2 siRNA for 30 hrs and then treated with 150 μM etoposide for 18 hrs. Apoptosis was quantified by caspase-3 activity and TUNEL staining as described above.

### B. Results

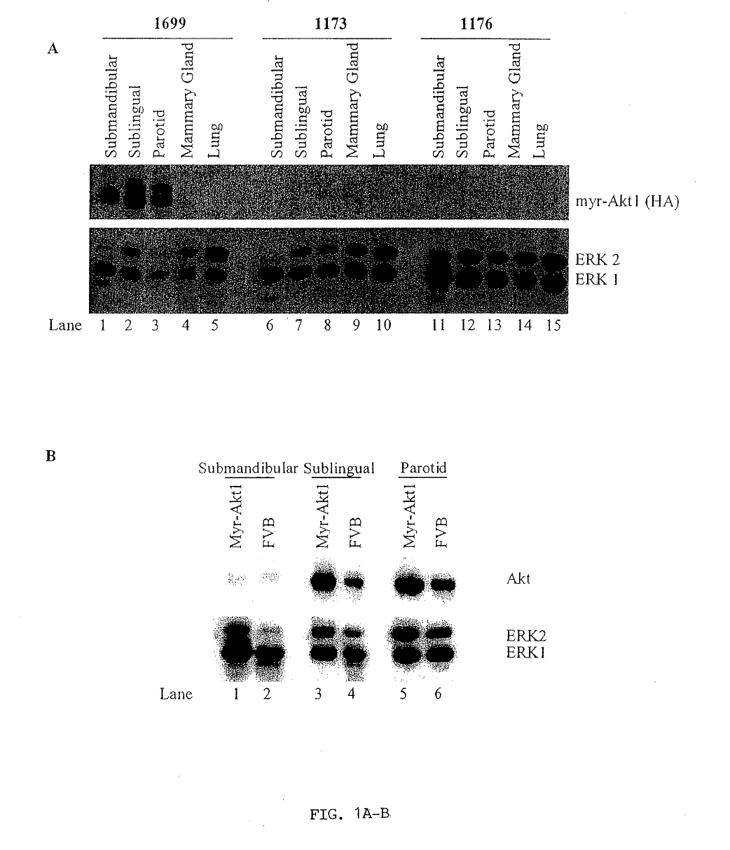

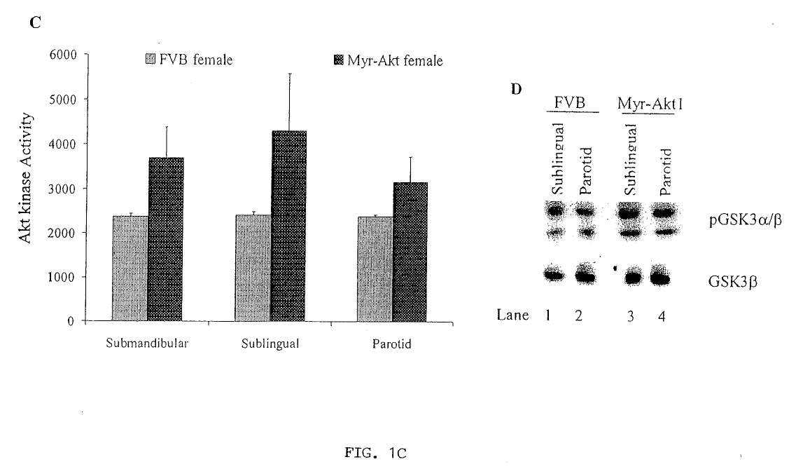

Identification of transgenic mice expressing myr-Akt1 in the salivary gland. Three different founder lines of myr-Akt1 transgenic mice were initially established that transmitted the transgene to their offspring and were designated 1173, 1176, and 1699. The effects of the myr-Akt1 transgene upon mammary gland biology in lines 1173 and 1176 has previously been described (Schwertfeger et al., 2003; Schwertfeger et al., 2001). The MMTV-LTR promoter is also expressed in the salivary gland (Henrard and Ross 1988); to determine whether the transgene was expressed in this tissue, the salivary glands (submandibular, sublingual, and parotid) were removed from four-week old female animals from each of the founder lines and immunoblot analysis was used to detect the transgene. Transgene expression in the salivary glands was shown to be significantly higher in the 1699 line (FIG. 1A; top panel; lanes 1-5) than in the two previously described founder lines (FIG. 1A; top panel; lanes 6-15). Consistent with our previous observation, two HA containing bands were detected (Schwertfeger et al., 2001) which may differ in their phosphorylation states (Schwertfeger unpublished results). Based upon the elevated level of transgene in the salivary glands, the 1699 line was chosen for further characterization. In female mice, total Akt protein levels were increased 1.7-fold in submandibular (FIG. 1B; lanes 1 and 2), 11.5-fold in sublingual (FIG. 1B; lanes 3 and 4) and 4.9-fold in parotid (FIG. 1B; lanes 5 and 6) salivary glands compared to FVB controls. Akt kinase activity was examined by measuring the phosphorylation of a substrate peptide (Limesand et al., 2003). In myr-Akt1 female mice, Akt kinase activity increased 1.56-fold in the submandibular gland, 1.78-fold in the sublingual gland and 1.33-fold in the parotid gland relative to endogenous Akt (FIG. 1C) with some variation between different transgenic animals. Although the kinase assay reveals a rather modest increase in Akt kinase activity, the increase is consistent with the increase in the amount of phosphorylated glycogen synthase kinase 3, a known Akt substrate, observed in the salivary glands of transgenic mice compared to wild-type mice (FIG. 1D). Therefore, both Akt protein and kinase activity were increased in the salivary glands of myr-Akt1 transgenic mice.

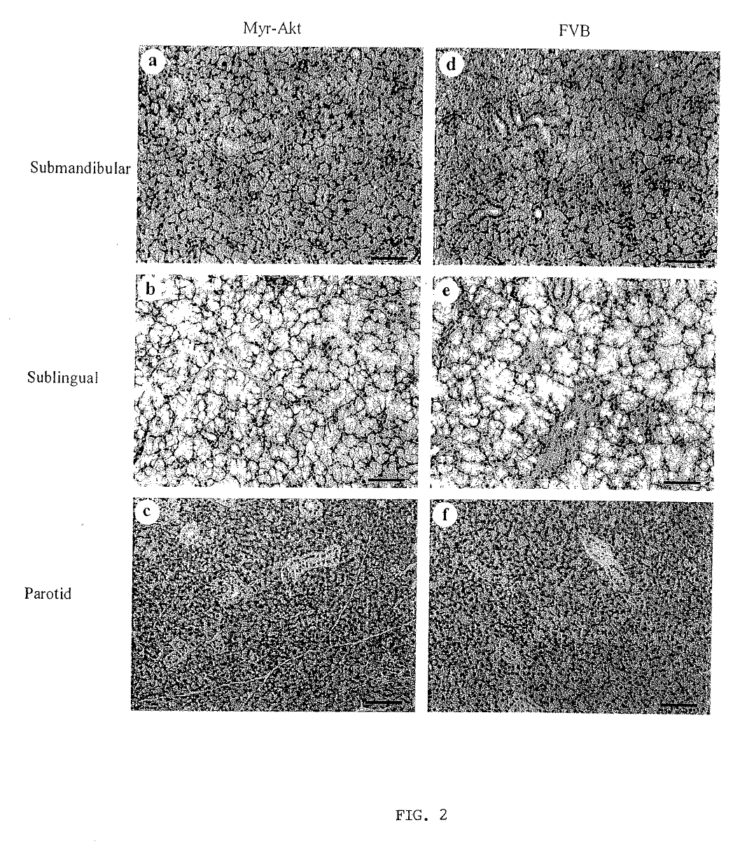

Histological analysis was performed on hematoxylin and eosin stained paraffin sections of control and transgenic mice to determine if transgene expression modified the structure and/or the development of the different salivary glands. Recent studies have reported increases in cell and tissue size in animals overexpressing Akt1 either in the pancreas or heart (Shioi et al., 2002; Tuttle et al., 2001). No gross morphological changes were detected in the myr-Akt1 salivary glands at low magnification (FIG. 2). To determine whether there was a change in cell size, glandular wet weights and DNA content were analyzed (Tables 1 and 2). Slight, but consistent (P values displayed in last line of table), increases in submandibular and parotid glandular wet weights relative to body weight (20% and 14% respectively) could be identified at four weeks of age (Table 1) and this size difference remained constant as the animals aged (>8 months; data not shown). Sublingual glands from myr-Akt1 mice were not significantly different in size from age matched FVB control glands possibly due to the small size of the tissue (12-16 mg). DNA content analysis was used to determine if increases in glandular size was due to an increase in the cell number or cell size (Table 2). No differences in total DNA content were detected in the myr-Akt1 submandibular, sublingual or parotid glands when compared to FVB salivary glands (P values displayed in last line of table), suggesting that the increase in wet weight of the submandibular and parotid glands resulted from a slight increase in cell volume or size that was not distinguished histologically.

TABLE 1
Salivary gland weight and salivary flow rate of myr-Akt1 transgenic mice

| | SMG/BW | SBL/BW | PAR/BW | Salivary flow rate |
|---|---|---|---|---|
| FVB | 0.342 ± 0.01 | 0.100 ± 0.004 | 0.354 ± 0.014 | 4.38 ± 0.76 |
| myr-Akt | 0.411 ± 0.015 | 0.105 ± 0.008 | 0.404 ± 0.011 | 4.85 ± 1.48 |
| t-test | 0.0007 | 0.56 | 0.013 | 0.78 |

Glandular wet weights were determined in four-week transgenic (N = 11) and control female animals (N = 9). Tissue weights (mg) were divided by total body weight (g) and multiplied by 100 to represent the glands' percentage of body weight. Abbreviations: BW, body weight; SMG, submandibular ± gland; SBL, sublingual gland; PAR, parotid gland. To determine salivary flow rates, mice were injected intraperitoneally with 0.25 mg carbachol per kg body weight. Induced saliva (N = 10/genotype) was collected into pre-weighed eppendorf tubes with a micropipette for five minutes and chilled on ice.

TABLE 2
DNA content of salivary glands

| | SMG | SBL | PAR |
|---|---|---|---|
| FVB | 1246 ± 83.7 | 2.5 ± 0.6 | 1372 ± 131.8 |
| myr-Akt | 1240 ± 68.5 | 3.6 ± 0.3 | 1330 ± 75.1 |
| t-test | 0.97 | 0.86 | 0.78 |

DNA content from salivary glands was determined using a PicoGreen assay kit. Total genomic DNA (mg) from four-week transgenic (N = 6) and control (N = 4) female animals is shown for each gland. Abbreviations: SMG, submandibular gland; SBL, sublingual gland; PAR, parotid gland.

Due to slight physical differences in the size of the salivary glands between FVB and the myr-Akt1 transgenic mice, the inventors examined whether changes in salivary flow rate and salivary protein composition could be detected. Saliva was collected from animals after carbachol stimulation over a five minute period; no differences in salivary flow rates were detected between myr-Akt1 transgenic mice and non-transgenic controls (Table 1, last column). No change in salivary protein composition could be detected between both groups of animals by SDS gel electrophoresis (data not shown). An important component of saliva is amylase, which is primarily produced by the parotid gland, and serves to hydrolyze starches to simple sugars (Zakowski and Bruns, 1985). Expression of amylase was analyzed in all three salivary glands by immunofluorescent staining and the distribution of amylase was very similar in the myr-Akt1 transgenic and FVB control animals (data not shown). Overall, the physiology of the myr-Akt1 salivary glands appears to be similar to FVB control animals and importantly no adverse defects in salivary gland function due to transgene expression could be detected.

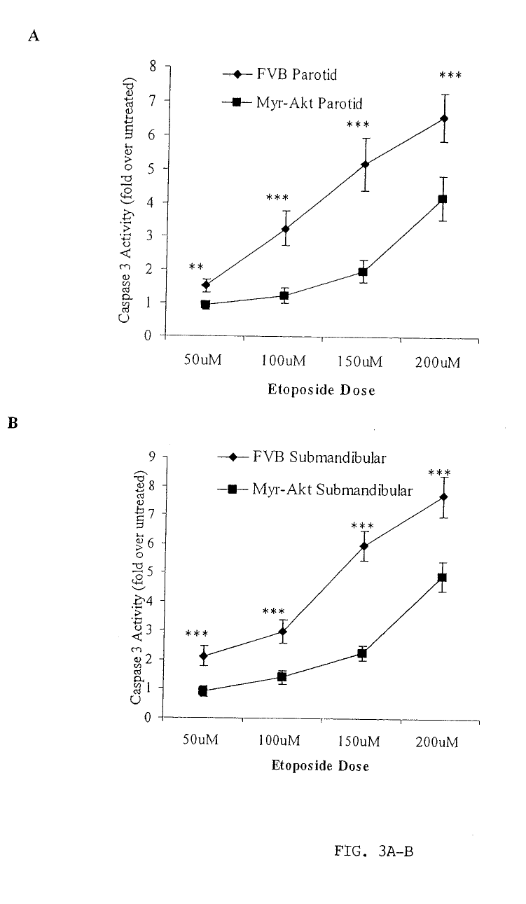

Suppression of apoptosis induced by DNA damage in primary salivary acinar cells from myr-Akt1 transgenic mice. Transfection of established cell lines with constitutively activated Akt suppresses apoptosis induced by a variety of stimuli (Dudek et al. 1997; Gibson et al., 1999; Henry et al., 2001; Kennedy et al., 1999; Kulik et al., 1997); therefore, the inventors wished to determine whether primary salivary acinar cells prepared from the myr-Akt1 transgenic mice were resistant to apoptotic stimuli. The inventors focused upon DNA damaging agents because they induce apoptosis in a p53-dependent manner and because of the clinical relevance of γ-irradiation-induced damage to salivary epithelial cells (Fox et al., 1985). Etoposide is a genotoxin that inhibits topoisomerase II resulting in double-stranded DNA breaks (van Maanen et al., 1988) and γ-irradiation induces both single and double-stranded DNA breaks (Jonathan et al., 1999). Primary cultures of parotid and submandibular cells were prepared as previously described (Limesand et al., 2003a; Limesand et al., 2003b; Quissell et al., 1997) and the proliferation of these cells was confirmed by incorporation of 5-Bromo-2′-deoxy-uridine (BrdU, data not shown). Etoposide-induced apoptosis was significantly reduced in primary salivary acinar cells from myr-Akt1 transgenic mice as indicated by activation of caspase-3 18 hrs after treatment (FIGS. 3A and B). Although etoposide did induce some apoptosis in the myr-Akt1 salivary acinar cells, the amount of caspase-3 activated was diminished significantly at all concentrations of etoposide examined (FIGS. 3A and B). Suppression of apoptosis in the myr-Akt1 expressing cells was confirmed by TUNEL (data not shown).

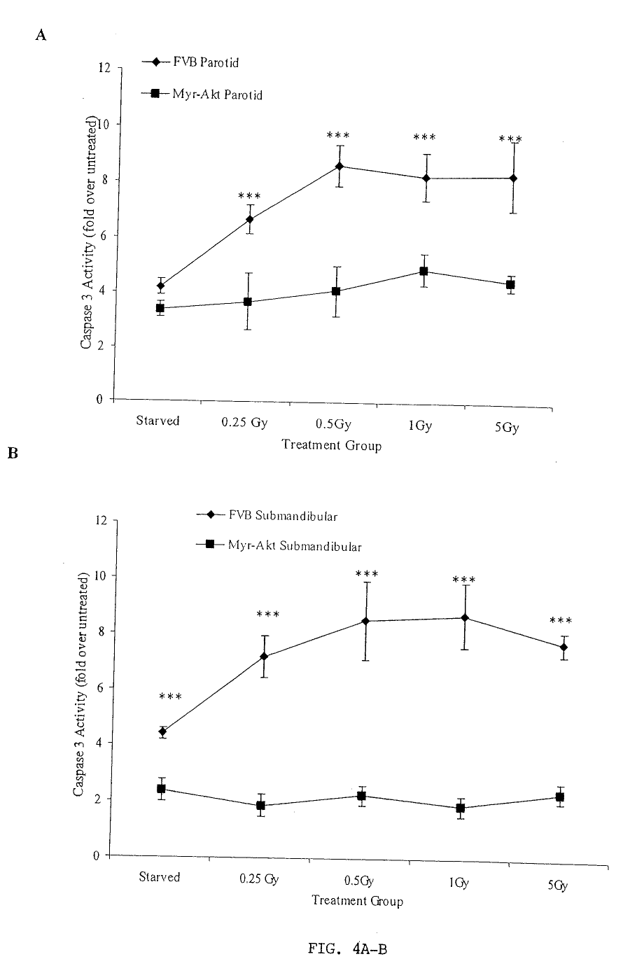

Exposure of primary salivary acinar cells from FVB control mice to γ-irradiation resulted in an induction of apoptosis (FIGS. 4A and B), and the extent of caspase-3 activation 24 hours after exposure was similar in both irradiated submandibular and parotid primary cells. In contrast, primary salivary acinar cells from myr-Akt1 transgenic mice were resistant to apoptosis based on quantitation of caspase-3 activity (FIGS. 4A and B). Even at the highest dose of irradiation used (5Gy), no caspase-3 activation was observed in either primary parotid or submandibular acinar cells from the myr-Akt1 transgenic mice (FIGS. 4A and B). Analysis of the response of primary parotid and submandibular cells to γ-irradiation using the TUNEL assay substantiated the results obtained with the caspase-3 assay (data not shown). Therefore, primary salivary acinar cells from myr-Akt1 transgenic mice are less responsive to apoptosis induced by two different DNA damaging agents, etoposide and γ-irradiation.

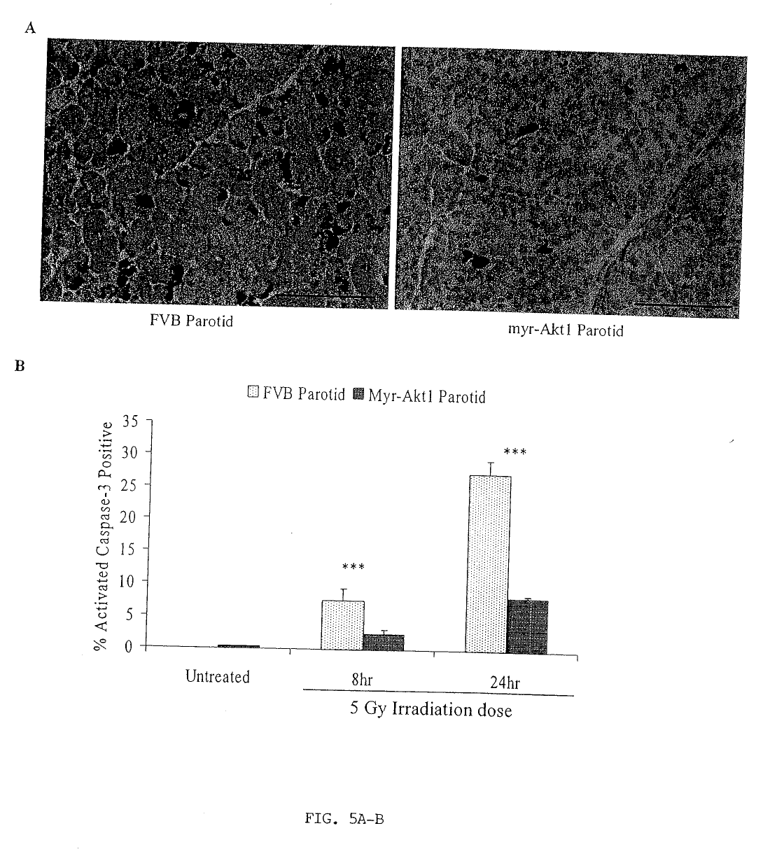

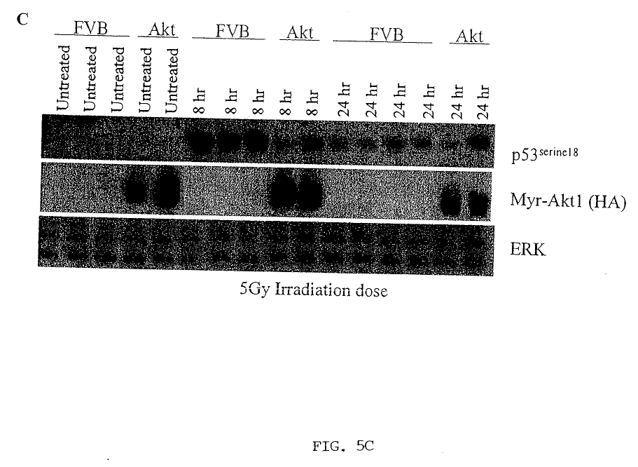

Diminished apoptosis in myr-Akt1 transgenic mice following in vivo γ-irradiation. It has been previously demonstrated that parotid salivary glands are sensitive to the effects of γ-irradiation resulting severe reductions in glandular function (Liem et al., 1996; Vissink et al., 1990). The head and neck region of FVB and myr-Akt1 transgenic mice was exposed to a single dose of 5Gy and parotid glands were collected 8 or 24 hours after γ-irradiation (FIGS. 5A-C). The number of apoptotic cells was quantitated by immunostaining with anti-active caspase-3 antibody (FIG. 5A) and graphed as a percent of total cells as determined with hematoxylin counterstaining (FIG. 5B). Apoptosis could be detected as early as 8 hrs post-irradiation with 7.4% of the cells positive for activated caspase-3 which increased to 27.4%, 24 hrs post-irradiation of FVB mice. In contrast, the level of apoptosis was diminished in the myr-Akt1 mice with 2.5% of the cells positive for activated caspase-3 8 hrs post-irradiation which increased to 8.2% at 24 hrs. These data demonstrate that expression of the myr-Akt1 transgene suppresses DNA damage-induced apoptosis in primary salivary acinar cells in vitro as well as in the salivary gland in vivo.

p53 activity induced by DNA damage results in an increase in total p53 protein levels and in the modification of p53 by phosphorylation and/or acetylation (Inoki et al., 2002; Li et al., 2003; Meek, 1999; Michael and Oren, 2003; Woods and Vousden, 2001). Phosphorylation of p53 on serine¹⁸ has been hypothesized to be required for its stabilization and activation following γ-irradiation (Woods and Vousden, 2001); however, this is not universal in all cell types or for the corresponding serine site in the mouse (Ashcroft et al., 1999; Blattner et al., 1999; Chao et al., 2003; Sluss et al., 2004). The inventors were interested in determining whether p53 was phosphorylated on serine¹⁸ in the salivary glands of FVB and myr-Akt1 transgenic mice exposed to irradiation. The parotid gland was dissected from FVB and myr-Akt1 transgenic mice 8 and 24 hrs post exposure to 5Gy γ-irradiation, as well as from unirradiated control mice. Exposure of FVB mice to γ-irradiation resulted in a dramatic increase in the level of serine¹⁸ phosphorylated p53 8 hrs after irradiation and the level of phosphorylated p53 dramatically decreased by twenty-four post-irradiation (FIG. 5C). In contrast the level of phosphorylated p53 was dramatically decreased 8 hrs post irradiation in both of the myr-Akt1 transgenic mice examined (FIG. 5C). At twenty-four hours post irradiation of the myr-Akt1 transgenic mice, phosphorylated p53 could be detected but it was not consistently elevated compared to that observed in the FVB mice at either 8 or 24 hrs post-irradiation. This suggests that the activation of p53 is dramatically reduced in the myr-Akt1 transgenic mice following exposure to γ-irradiation.

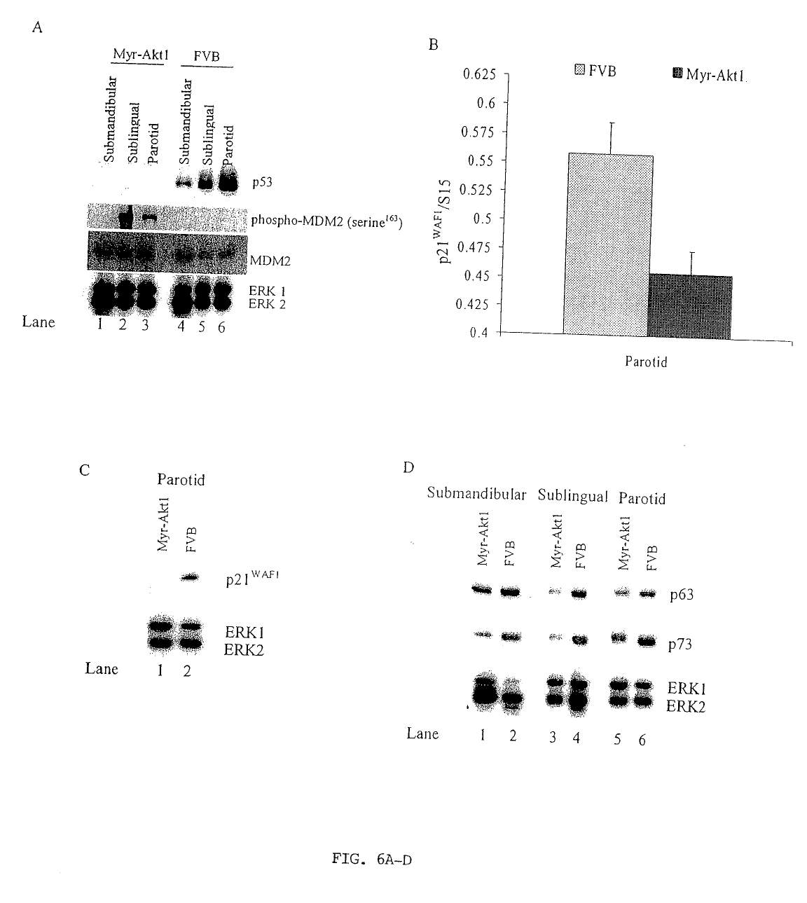

Basal expression of p53 family members is reduced in salivary glands of myr-Akt1 transgenic mice. As noted above, p53 plays a significant role in apoptosis induced by DNA damage. The results shown in FIG. 5C suggest that activation of p53 is blocked or diminished in the myr-Akt1 transgenic mice and there are many mechanisms that could account for this change. Akt has been reported to phosphorylate and activate MDM2 causing MDM2 to translocate to the nucleus where it degrades p53 (Ogawara et al., 2002; Oren et al., 2002). For this reason the inventors determined the levels of total p53 in salivary gland tissue lysates isolated from untreated four-week-old myr-Akt1 and FVB female mice (FIG. 6A). Expression of constitutively activated Akt1 resulted in dramatically reduced levels of total p53 (FIG. 6A, top panel, lanes 1-3), which correlated with an increase in the phosphorylation of MDM2 at serine¹⁶³, a putative Akt phosphorylation site (FIG. 6A, second panel). No differences in p53 RNA expression in the salivary glands could be detected between untreated FVB and myr-Akt1 female mice by microarray analysis or quantitative RT/PCR (data not shown). Levels of endogenous total MDM2 (FIG. 6A, third panel) are also increased in all three salivary glands from transgenic mice suggesting that phosphorylation of MDM2 may increase its stability (Feng et al., 2004). The decrease in total basal p53 levels clearly explains why there is a reduced level of serine¹⁸ phosphorylated p53 in the salivary gland of irradiated myr-Akt1 transgenic mice (FIG. 5C).

To determine whether the decrease in p53 protein levels led to a decrease in the expression of a p53 responsive gene, the inventors used quantitative RT/PCR to determine the basal concentration of p21^{WAF1} RNA in parotid salivary glands of FVB and myr-Akt1 transgenic mice (FIG. 6B). Expression of constitutive activated Akt1 in the parotid glands resulted in significantly diminished levels of p21^{WAF1} mRNA (FIG. 6B). Consistent with the changes in p21^{WAF1} RNA level, there is a decrease in p21^{WAF1} protein (FIG. 6C; top panel; lane 1). Significant reductions in total p53 protein levels along with decreased expression of p21^{WAF1} mRNA in the salivary glands of transgenic mice indicate that Akt-dependent regulation of p53 by MDM2 might function in vivo in a manner similar to that shown in vitro using transfected cells (Mayo and Donner, 2001; Ogawara et al., 2002).

Recently two p53 homologues, p63 and p73, have been described and expression of these homologues is required for p53-dependent apoptosis (Flores et al., 2002). However, the importance of p63 and p73 in p53-dependent processes may not be universal in all tissue types (Senoo et al., 2004). It is intriguing to note that loss of either p63 or p73 cooperates with the loss of p53 in accelerating tumor development at many sites including the salivary gland (Flores et al., 2005). Clearly these studies suggest that the combined role of p53, p63 and p73 is complex and may be cell type or stimulus type specific. The inventors also evaluated the levels of p63 and p73 by immunoblot analysis to complete our analysis of the p53 family members in the salivary glands of myr-Akt1 transgenic mice. Densitometric analysis of total p63 levels revealed a 43% reduction in the submandibular glands of myr-Akt1 transgenic mice (FIG. 6D; top panel; lane 1), a 78% reduction in the sublingual glands of myr-Akt1 transgenic mice (FIG. 6D; top panel; lane 3) and a 36% reduction in the parotid glands of myr-Akt1 transgenic mice (FIG. 6D; top panel; lane 5) when compared to the respective FVB control salivary glands (FIG. 6D, lanes 2, 4, and 6 respectively). Total p73 levels also were reduced in the myr-Akt1 transgenic mice; a 61% reduction in the submandibular glands (FIG. 6D; middle panel; lane 1), an 83% reduction in the sublingual glands (FIG. 6D; middle panel; lane 3) and a 48% reduction in the parotid glands (FIG. 6D; middle panel; lane 5) when compared to the respective FVB control salivary glands (FIG. 6D, lanes 2, 4, and 6 respectively). The antibodies used to detect p63 and p73 should detect all isoforms; however, only one band corresponding to full-length p63 (˜63Kd) or p73 (˜73Kd) was detected. The reduction in p63 and p73 proteins appears to result from a decrease in the expression of these genes in the salivary gland of myr-Akt1 transgenic mice as determined by quantitative real-time RT/PCR (FVB vs. myr-Akt, two sample t-test, P<0.05 for RNA or protein concentration; real-time data not shown). To date there is no evidence that MDM2 regulates the degradation of either p63 or p73 (Wang et al., 2001).

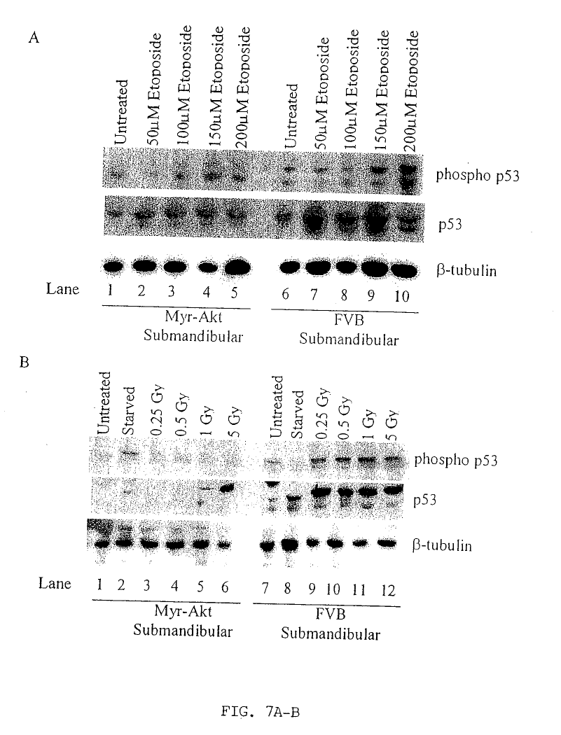

Diminished levels of total p53, phosphorylated p53, and p53 target genes in myr-Akt1 salivary acinar cells following DNA damage. In order to evaluate the affect of myr-Akt1 expression on p53 activation following DNA damage, the inventors used primary salivary acinar cells. Phosphorylation of p53 on serine¹⁵ has been hypothesized to be required for its stabilization following γ-irradiation (Woods and Vousden, 2001); however, this is not universal in all cell types or for the corresponding serine site in the mouse (Ashcroft et al., 1999; Blattner et al., 1999; Chao et al., 2003; Sluss et al., 2004). Levels of total p53 and phosphorylated p53^{serine18} were determined by immunoblot analysis of etoposide and γ-irradiation treated primary salivary acinar cells (FIG. 7). Total p53 levels increased following etoposide treatment of FVB primary cells at all concentrations of etoposide (FIG. 7A; top panel; lanes 7-10); however phosphorylation of p53 at serine¹⁸ could only be detected at 150 and 200 μM of etoposide (FIG. 7A; top panel; lanes 9 and 10). This difference may reflect a difference in the sensitivity of the anti-p53 antibody versus the anti-phospho-serine¹⁸ antibody, or it may indicate that post-translational modifications of p53 other than phosphorylation of serine¹⁸ also may be important in stabilizing p53 following DNA damage. In contrast, etoposide treatment of myr-Akt1 primary cells did not result in demonstrable increases in total p53 or p53 phosphorylation at serine¹⁸ (FIG. 7A, lanes 2-5).

Treatment of FVB derived submandibular salivary acinar cells with γ-irradiation also lead to an increase in p53 phosphorylation and an increase in the level of total p53 as compared to untreated or starved salivary acinar cells at all doses of irradiation (FIG. 7B, lanes 7 and 8 versus 9-12). The amounts of phosphorylated p53^{serine18} were dramatically reduced in irradiated primary salivary acinar cells from myr-Akt1 transgenic mice compared to irradiated cells from FVB mice (FIG. 7B; top panel; lanes 3-6 versus 9-12). An increase in total p53 protein was only detected after treatment of myr-Akt1 primary salivary acinar cells with the highest dose of irradiation examined (5Gy; FIG. 7B, middle panel, lane 6); however this did not correspond with an increase in serine¹⁸ phosphorylated p53 (FIG. 7B, top panel, lane 56). This also may indicate that post-translational modifications of p53 other than phosphorylation of serine¹⁸ may be important in stabilizing p53 following DNA damage. Alternatively a decrease in transgene expression may occur at high doses of γ-irradiation. Similar results were obtained with primary cultures derived from either the parotid or the submandibular glands. In contrast to the myr-Akt1 tissue lysates, the expression of p53 can be detected in the primary salivary acinar cells isolated from the myr-Akt1 transgenic mice suggesting that placing these cells into culture may alone be enough stress to induce stabilization of p53 (Compare FIGS. 6A-D and 7A-B). Expression of the myr-Akt1 transgene; however, still results in lower levels of p53 protein levels following DNA damage when compared to FVB controls. The data presented in FIGS. 6A-D and 7A-B suggest that the down-regulation of p53, perhaps mediated by Akt-dependent phosphorylation of MDM2, may be responsible for the resistance of the myr-Akt1 salivary acinar cells to DNA damage-induced apoptosis.

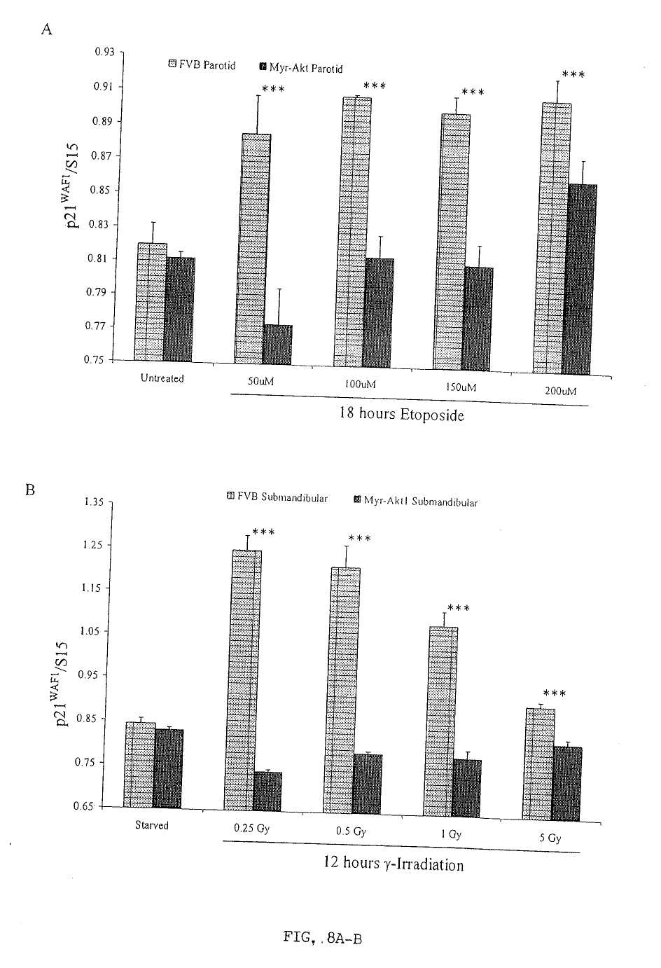

Activation of p53 has been associated with the transcription of numerous genes involved in cell cycle arrest such as p21^{WAF1} (Bouvard et al., 2000), 14-3-3σ (Vogelstein et al., 2000) and GADD 45 (Maity et al., 1994) as well as proteins that regulate apoptosis such as Bax (Miyashita et al., 1994), PUMA (Yu et al., 2001), Noxa (Oda et al., 2000) and PERP (Attardi et al., 2000). The best characterized p53 target gene that is induced following γ-irradiation of a wide variety of cells is the cell cycle arrest gene p21^{WAF1} (Brugarolas et al., 1995; Chin et al., 1997). Expression of p21^{WAF1} can also be stimulated by p63 and p73 and thus may represent a universal target for all p53 family members (Dietz et al., 2002). p21^{WAF1} RNA concentrations were determined by real-time RT/PCR (Wan and DeGregori, 2003) following treatment of cells with different doses of etoposide (FIG. 8A). The amount of p21^{WAF1} RNA was normalized to the ribosomal protein S15 RNA and was found to be significantly reduced in primary salivary acinar cells from myr-Akt1 mice compared to FVB control cells 18 hours after etoposide treatment (FIG. 8A). The inventors also evaluated the induction of Bax by quantitative RT/PCR. Bax expression increased in FVB control cells treated with higher concentrations of etoposide (100-200 μM) while myr-Akt1 primary cells demonstrated no induction of Bax expression following etoposide treatment (data not shown).

Expression of p21^{WAF1} also was determined twelve hours following treatment of primary salivary acinar cells from FVB and myr-Akt1 transgenic mice with 0.25-5 Gy γ-irradiation. (FIG. 8B). There was no statistical difference in the abundance of p21^{WAF1} RNA between starved myr-Akt1 or starved FVB primary submandibular acinar cells. In comparison to starved controls, γ-irradiation of FVB primary salivary acinar cells significantly increased the amount of p21^{WAF1} RNA at each dose of irradiation. However, the levels of p21^{WAF1} following γ-irradiation of myr-Akt1 cells were not increased. These data suggest reduced stabilization and phosphorylation of p53 on serine¹⁸ in myr-Akt1 primary salivary acinar cells prevents induction of downstream gene targets such as p21^{WAF1} following γ-irradiation.

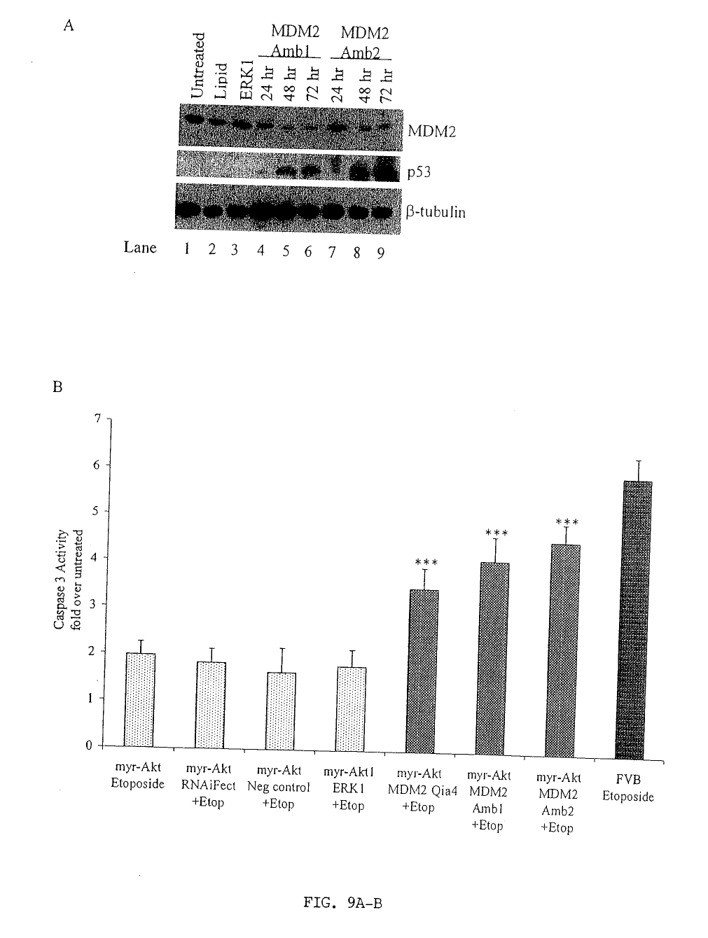

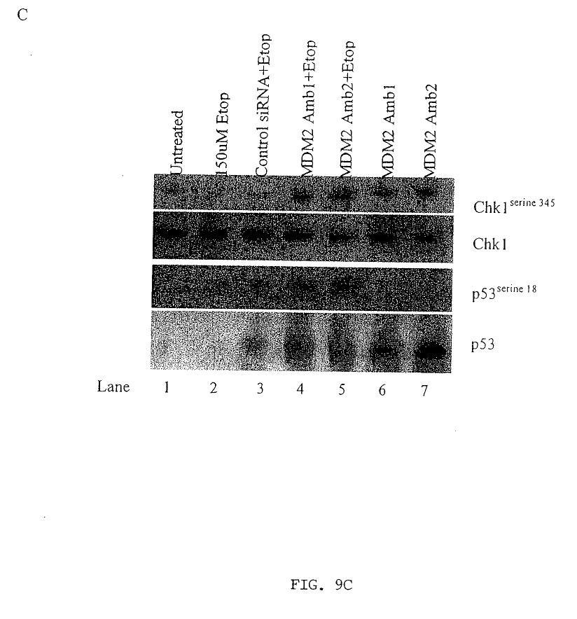

MDM2 is required for the ability of myr-Akt1 to suppress apoptosis. It has been previously demonstrated in tissue culture cells transfected with constitutively activated mutants of Akt1, that MDM2 is a substrate for Akt (Ogawara et al., 2002; Oren et al., 2002). To determine whether MDM2 is required for suppression of apoptosis in myr-Akt1 salivary acinar cells, the inventors depleted these cells of MDM2 using siRNA molecules specific for murine MDM2. Lipid transfection reagent alone or transfection with an siRNA that targeted ERK1 had no effect on the total amount of MDM2 protein (FIG. 9A, top panel, lanes 1-3). The ability of two different siRNA molecules to reduce the level of MDM2 protein 24, 48, and 72 hours after transfection of primary salivary acinar cells is shown in FIG. 9A (top panel, lanes 4-9). Reduction of MDM2 expression correlated with the increase in total p53 protein at 48 and 72 hours after transfection with siRNA targeting MDM2 (FIG. 9A, middle panel, lanes 5, 6, 8, and 9).

Primary salivary acinar cells isolated from myr-Akt1 transgenic mice were transfected with various siRNA molecules for 30 hrs prior to treatment with etoposide for 18 hours. The extent of caspase-3 activation was used to quantitate induction of apoptosis. No significant differences were detected between primary salivary acinar cells from myr-Akt1 mice transfected with control siRNA molecules and etoposide treated primary salivary acinar cells expressing myr-Akt1 (FIG. 9B). In contrast, three siRNA molecules directed against MDM2 (MDM2 Qia4, MDM2 Amb1, and MDM2 Amb2) rendered primary salivary acinar cells from myr-Akt1 mice sensitive to etoposide-induced apoptosis (FIG. 9B). In addition, there was no statistical difference between the amount of caspase-3 activity induced in etoposide treated FVB control primary acinar cells and the etoposide treated myr-Akt1 primary cells transfected with the MDM2 Amb2 siRNA (FIG. 8B, bar 7 vs. 8; P≦0.49). The inventors also investigated the activation of ATM and Chk1 using phosphorylation specific antibodies following etoposide treatment (FIG. 9C). These key molecules in the DNA damage response lie upstream of p53 activation and may also be modified by Akt (King et al., 2004; Puc et al., 2005; Viniegra et al., 2005). Etoposide treatment of primary acinar cells from myr-Akt1 transgenic mice resulted in the phosphorylation of ATM at serine¹⁹⁸¹ regardless of whether the cells had been transfected with siRNA that targeted MDM2 (data not shown). This suggests that ATM is activated in the primary cells from myr-Akt1 transgenic mice following treatment of the cells with DNA damaging agents. Increased levels of Chk1 that is phosphorylated on serine³⁴⁵ were observed in myr-Akt1 primary salivary cells transfected with siRNA targeting MDM2 (FIG. 9C; lanes 4-7) with a slight increase (34%) in the amount of phosphorylation following etoposide treatment relative to that seen in the cells not treated with etoposide (FIG. 9C; lanes 4-5 vs. lanes 6-7). The total levels of Chk1 were largely unaffected by treatment of these primary cells. A higher level of p53 phosphorylation on serine¹⁸ was detected in myr-Akt1 primary cells transfected with siRNA targeting MDM2 following etoposide treatment when compared to the level of phosphorylated p53 observed in cells treated with the control siRNA (FIG. 9C; lane 3 versus lanes 4-5). Reducing the level of MDM2 by siRNA also significantly increased total p53 protein levels (FIG. 9C, bottom panel, lanes 4-7) however, phosphorylation of p53 on serine¹⁸ was only observed following etoposide treatment (FIG. 9C, 3^{rd} panel, lanes 4-5). Moreover, these data provide further evidence that MDM2 is required for the ability of myr-Akt1 to suppress etoposide-induced apoptosis by regulating the level of total p53.

C. Discussion

Tissue homeostasis results from a balance in the amount of cellular proliferation and programmed cell death or apoptosis. Irradiation of the head and neck region of rodents has been shown to result in salivary gland hypofunction similar to that observed in humans receiving ionizing irradiation (Liem et al., 1996; Nagler, 2003). In the rat animal model, salivary gland function is diminished 50-70% six to nine months following exposure to γ-irradiation; however, previous studies reported that only 2% of cells exhibited condensed nuclei on H&E stained sections (Nagler et al., 1998; Paardekooper et al., 1998). Because of the disparity between the extent of apoptosis and the extent of salivary gland hypofunction, some authors have concluded that apoptosis is not causally related to salivary gland hypofunction (Paardekooper et al., 1998). Although the apoptotic index observed in a tissue reflects the balance between the induction of apoptosis and the clearance of apoptotic cells, our data indicates that there is far more apoptosis 24 hours following irradiation with a single dose of 5 Gy then previously suggested by quantitating the number of condensed nuclei. Better understanding of γ-irradiation-induced damage to salivary glands is clinically relevant to improving the care of patients undergoing treatment for head and neck cancer.

The ability of Akt to suppress apoptosis induced by various stimuli is well established (Dudek et al., 1997; Gibson et al., 1999; Henry et al., 2001; Kennedy et al., 1999; Kulik et al., 1997). Primary salivary acinar cells isolated from myr-Akt1 transgenic mice consistently had lower levels of DNA damage-induced apoptosis compared to control cells isolated from age-matched FVB mice. Importantly, this suppression of apoptosis by myr-Akt1 was also observed in vivo following exposure of the head and neck region to γ-irradiation treatment. Following DNA damage, p53 undergoes several post-translational modifications and the stability of the p53 protein increases dramatically (Meek, 1999). The inventors determined the levels of total p53 and serine¹⁸ phosphorylated p53 by immunoblot analysis and quantitated the levels of p53-responsive genes (p21^{WAF1} and Bax) by quantitative RT/PCR. Levels of total p53 and phosphorylated p53 (serine¹⁸) were increased in primary cells from FVB mice following exposure to either etoposide or γ-irradiation (FIG. 6), resulting in the induction of p21^{WAF1} and Bax expression in these cells (FIG. 7 and data not shown). The induction of p21^{WAF1} was observed following low doses of etoposide or γ-irradiation and this reaction could contribute to the sensitivity of the salivary glands to ionizing radiation in vivo. In contrast, primary salivary acinar cells from myr-Akt1 transgenic mice were less sensitive to etoposide and γ-irradiation-induced apoptosis, and had reduced levels of total p53, phosphorylated p53, p21^{WAF1} RNA and Bax RNA following exposure to DNA damaging agents.

It has recently been reported that Akt negatively regulates the levels of p53 protein via activation of MDM2 (Mayo and Donner, 2001; Ogawara et al., 2002; Oren et al., 2002). Normally, p53 is maintained at low levels in the cell due to its short half-life, which is in part regulated by MDM2 (Oren, 1999). MDM2 is a E3 ubiquitin ligase that targets p53 for ubiquination and subsequent degradation by the proteosome (Michael and Oren, 2003; Momand et al., 2000). Analysis of the Akt/MDM2/p53 pathway has largely been accomplished by transfection of established cell lines with activated mutants of Akt. In transgenic mice expressing constitutively activated Akt1, there is an increase in total MDM2 protein and MDM2 phosphorylation at serine¹⁶³ which correlates with a dramatic reduction in total p53 protein suggesting that Akt regulates p53 protein expression in vivo. The inventors extended this observation to show that MDM2 is a critical substrate of Akt in the suppression apoptosis induced by DNA damage in primary salivary acinar cells from myr-Akt1 transgenic mice. It is interesting to note that phosphorylation of Chk1 at serine³⁴⁵, the putative phosphorylation site for ATM/ATR, occurs under conditions of reduced MDM2 expression in myr-Akt1 cells. This may indicate a feed-back mechanism by which phosphorylation of Chk1 is dependent upon the level of total p53 protein levels.

It has been suggested that the p53 homologs p63 and p73 are required for p53-dependent apoptosis induced following DNA damage; however p63 and p73 are not required for p53-dependent apoptosis in T cells (Flores et al., 2005; Senoo et al., 2004). Full-length versions of p73 and p63 (termed TA to indicate presence of a transactivation domain) have been shown to induce apoptosis. Deletion of the N-terminal transactivation domain of p73 or p63, through utilization of the second transcriptional start site (termed ΔN), produces a protein that serves as a dominant negative molecule that suppresses action of the respective full-length isoforms, as well as p53 (Yang and McKeon, 2000). Loss of p63 and p73 cooperate with the loss of p53 to accelerate tumor formation suggesting that p63 and p73 may function as tumor suppressors in some tissues, including salivary glands (Flores et al., 20050. For these reasons it was important to examine the levels of all p53 family members in the salivary glands of myr-Akt1 transgenic mice. The basal levels of p53, p63 and p73 were all reduced in myr-Akt1 mice. Regulation of p73 activity may occur by MDM2-dependent translocation without degradation, or may involve another putative Akt substrate, Yes associated protein (YAP) (Basu et al., 2003; Strano et al., 2001), however neither of these molecules can explain the reduced level of p73 RNA the inventors have observed. Modulation of p63 protein levels by activated Akt has not been previously observed and can not be explained by the phosphorylation of MDM2 since p63 and MDM2 do not interact (Wang et al, 2001). The inventors are currently conducting additional studies to understand the regulation of p63 by Akt and to investigate whether YAP is phosphorylated in the salivary glands of myr-Akt1 transgenic mice.

Significant efforts have focused on the identification of Akt substrates that suppress apoptosis in tissue culture cells (Brunet et al., 1999; Cardone et al., 1998; Kandel and Hay, 1999; Kennedy et al., 1999; Mitsiades et al., 2002). Analysis of transgenic mice that express activated Akt1 has confirmed the importance of some of these substrates. For example, Wendel et al. demonstrated the chemoresistance of a B-cell lymphoma that overexpresses Eμ-Myc and activated Akt1 was dependent on mTOR and eIF4E, and an mTOR inhibitor, rapamycin, reversed the Akt-induced chemoresistance in these cells (Wendel et al., 2004). This result was somewhat surprising given the number of Akt substrates regulating apoptosis and emphasizes that the critical substrates for Akt may differ with the stimuli and cell type examined. The inventors' analysis of activated Akt1 overexpression in vivo has revealed a universal reduction in the expression of the p53 family of proteins in salivary acinar cells; although the mechanism underlying this observation may differ for each p53 family member. It is well known that mutations in p53 and/or overexpression of MDM2 occurs in many cancers and numerous studies have focused on the dysregulation of p53 in cancer (Lane and Lain, 2002). Disruption of the MDM2/p53 regulation pathway shows great promise in re-activating wildtype p53 in cancer cells (Vassilev et al., 2004), as well as sensitizing tumors to radiation therapy (Perry, 2004). Our studies have demonstrated that MDM2 is a critical substrate of Akt in suppression of apoptosis caused by DNA damage. These studies also suggest that the resistance of tumors expressing activated Akt to chemotherapy and radiation therapies could be reversed by targeting MDM2's function or expression. Future studies will determine if targeted disruption of MDM2 in the salivary glands sensitizes myr-Akt1 transgenic mice to γ-irradiation in vivo.

### Example 2

### A. Materials and Methods

Mice. Myr-Akt1 transgenic mice were generated using standard techniques as described previously by the Transgenic Mouse Core of the University of Colorado Cancer Center (Schwertfeger et al., 2001). Genomic tail DNA was extracted and transgenic mice were identified using PCR as previously described (Schwertfeger et al., 2001). Non-transgenic littermates were used as controls in all experiments. Animals were maintained and treated in accordance with protocols approved by the University of Colorado Health Sciences Center Laboratory Animal Care and Use Committee.

γ-irradiation treatment. For in vivo experiments, four-week old female FVB and myr-Akt1 transgenic mice were anesthetized with avertin (0.4-0.6 mg/gm, IP) and the head and neck region was exposed to ionizing radiation using a RS 2000 Biological Irradiator (Humphries et al., 2006). The rest of the body was shielded with 6 mm lead foil and sheets to avoid systemic effects of γ-irradiation. Mice completely shielded with lead exhibited no adverse effects of radiation suggesting that the body is not exposed to radiation. For IGF1 injections, mice were anesthetized with avertin and tails were warmed in water to allow dilation of blood vessels. Mice were injected with a total volume of 100 μl of IGF1 diluted in sterile PBS and immediately placed into the radiation chamber.

Histology. Tissues were fixed in 10% neutral buffered formalin, and then embedded in paraffin. Tissue sections were cut at 4 μm and processed for standard staining with hematoxylin and eosin by the Histology Service of the Department of Pathology at the University of Colorado School of Medicine. Tissue sections were observed by standard light microscopy and photomicrographs were taken with an Olympus BX51 microscope with Spot Diagnostic imaging software.

Activated caspase-3 staining. Salivary glands were removed at various time points post-irradiation, fixed in 10% neutral buffered formalin, and then embedded in paraffin. Slides were stained for activated caspase-3 (Cell Signaling #9661; Beverly, Mass.) as previously described (Humphries et al., 2006). Cell counts were performed on a minimum of five fields of view per slide from three mice (total cells counted ranged from 1800-2500 per mouse).

Saliva collection. Mice were injected intraperitoneally with 0.25 mg carbachol per kg body weight (Lin et al., 2001). Saliva was collected immediately following injection for five min and chilled on ice as previously described (Limesand et al, manuscript submitted).

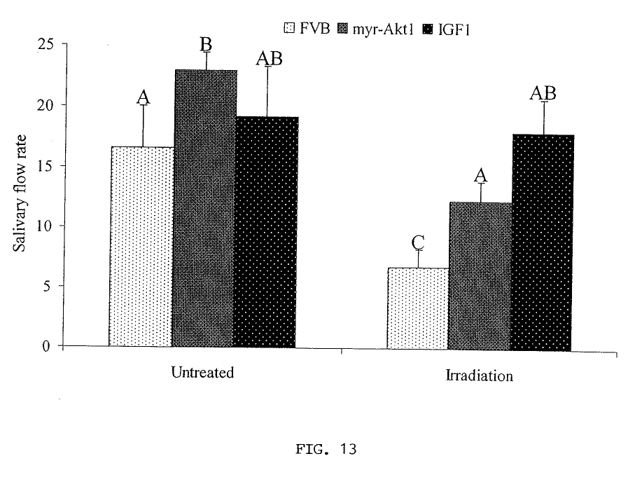

Statistics. Statistical evaluation for multiple comparisons (FIG. 13) was performed in SAS using general linear means and the least squares means for effect to control for Type I errors. All other statistical calculations were performed using a two sample t-test in Microsoft Excel.

### B. Results and Discussion

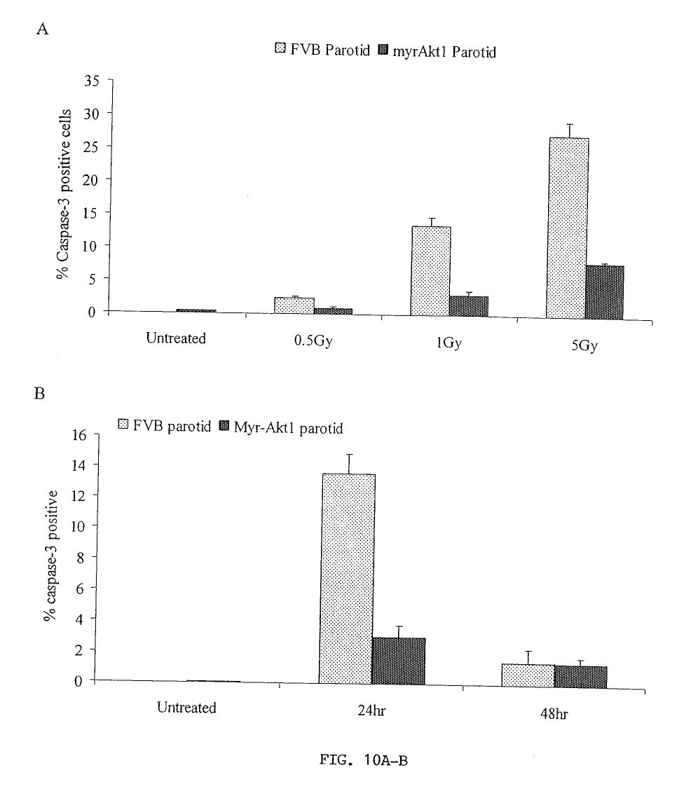

The inventors have described the phenotype of transgenic mice that express the myr-Akt1 transgene in either the mammary gland (Schwertfeger et al., 2001) or the salivary gland (Limesand et al, unpublished). In this study, they evaluated the amount of apoptosis in the parotid salivary gland following exposure of the head and neck region of control FVB and myr-Akt1 transgenic mice to increasing doses of γ-irradiation. Apoptosis was quantified using immunohistochemical staining for activated caspase-3. Twenty-four hours after treatment, a significant level of apoptosis was detected in FVB control mice (FIG. 10A) exposed to 1 and 5Gy doses of radiation. Because therapeutic doses administered to patients are kept below 2Gy per exposure, we decided to evaluate apoptosis and salivary function following a single dose of radiation over a lower range (0.5, 1, 2 and 5Gy/dose). The number of apoptotic cells was quantitated at both 24 and 48 hrs following exposure. The highest level of apoptosis in the irradiated salivary glands was observed at 24 hrs post radiation treatment (FIG. 10B); with very few apoptotic cells detected at 48 hours following irradiation. Approximately 13% of the salivary cells were positive for activated caspase-3 following exposure to 1 Gy and 27% were caspase-3 positive following exposure to 5 Gy γ-irradiation (FIG. 10A) Expression of myr-Akt1 dramatically reduced the number of caspase-3 positive cells at both doses of irradiation; approximately 3% of salivary acinar cells were apoptotic following exposure to 1Gy and approximately 8% of the cells were positive for active caspase-3 following a single exposure to 5Gy γ-irradiation (FIG. 10A). Akt suppressed the early induction of apoptosis with a return to basal levels by 48 hrs suggesting that Akt diminishes the overall cell death in the tissue rather than delaying cell death. These data are consistent with numerous students that have demonstrated that expression of a constitutively active mutant of Akt is able to suppresses apoptosis induced by many different stimuli both in vitro and in vivo (Datta et al., 1999; Coffer et al., 1998; Kandel & Hay, 1999).

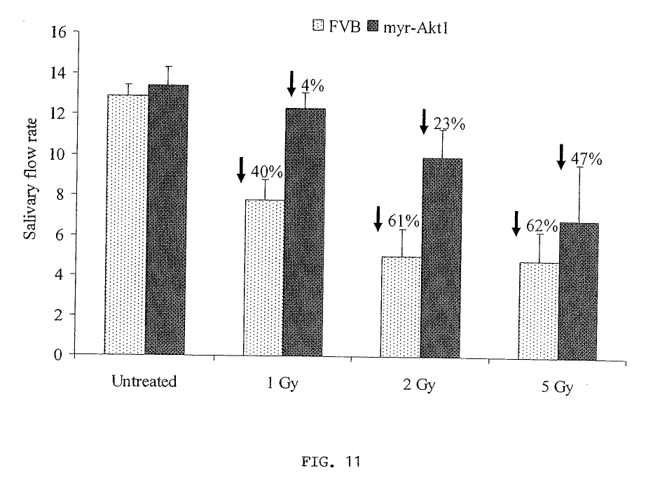

Radiation-induced suppression of salivary function has been previously reported to occur almost immediately after exposure, reach a minimum at three days, and remain decreased for 180 days in the rat (Coppes et al., 2001). The significant differences in apoptosis detected between myr-Akt1 transgenic mice and FVB control mice (FIGS. 10A-B) stimulated us to ask whether there was a difference in salivary flow rate between these two strains of mice. A significant decrease (40-62%) in salivary flow was detected in FVB control mice at each dose of radiation examined (FIG. 11). In myr-Akt1 transgenic mice, the decrease in salivary flow rate was less severe (4-47%) with a marked improvement in salivary output at the lower doses of radiation (1-2Gy). These data clearly indicate that, contrary to previous conclusions by other investigators, the extent of apoptosis induced in the salivary glands by head and neck irradiation may be causally related to salivary gland dysfunction.

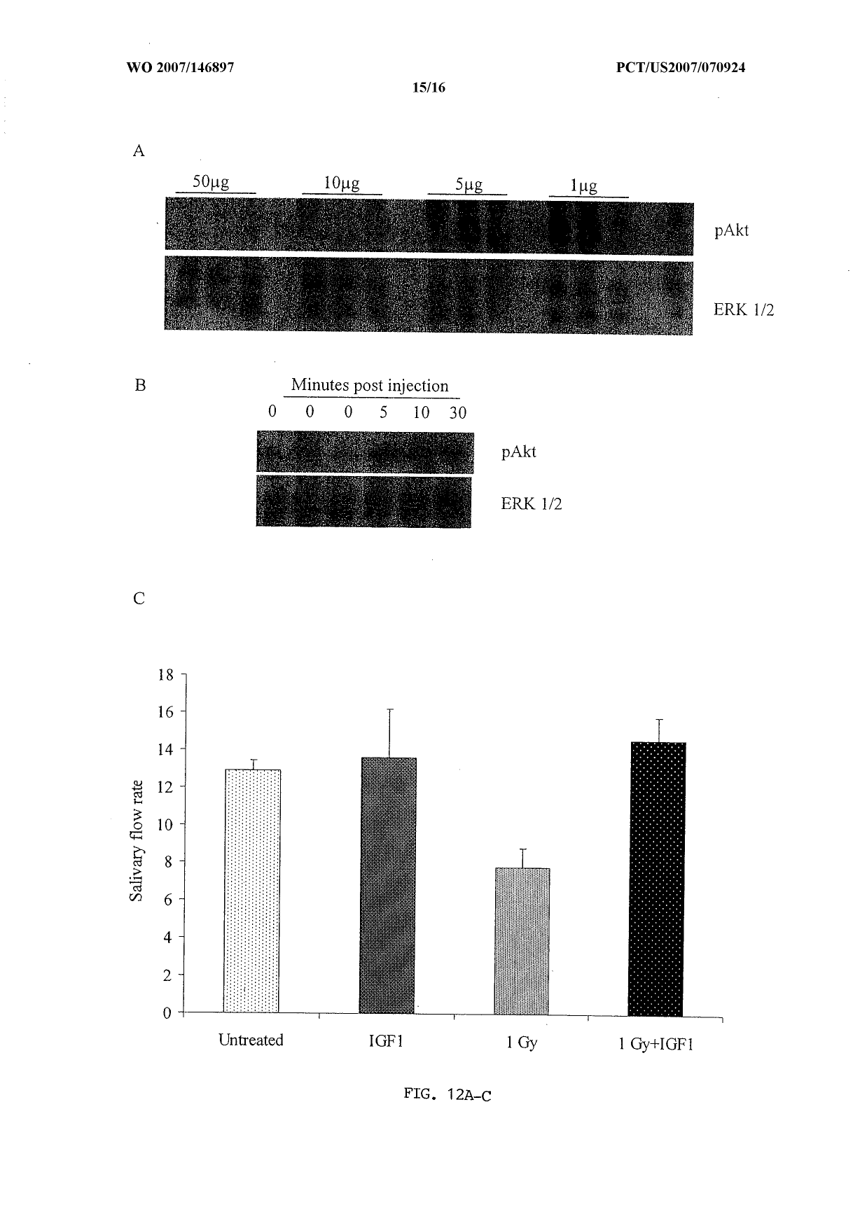

The inventors have previously shown that IGF1 induces activation of Akt in salivary acinar cells and suppresses apoptosis induced by etoposide (Limesand et al., 2003). Stimulation of cells with both IGF1 and EGF produced a synergistic activation of Akt and suppression of apoptosis (Limesand et al., 2003). They therefore determined whether intravenous injections of IGF1 into the tail vein of mice would activate endogenous Akt in the salivary gland similar to a previous study that had demonstrated that intravenous injection of IGF1 could induce acute activation of Akt in the mammary gland of treated mice (Lee et al., 2003). Mice were injected with 1, 5, 10, or 50 μg recombinant IGF1 and the activation of Akt was examined in the salivary gland five minutes after injection. Injection of 1 μg and 5 μg IGF1 results in the activation of Akt as determined by immunoblotting with anti-phospho-Akt (threonine⁴⁷³) antibody (FIG. 12A). It is not clear why injection of doses of 10 and 50 μg IGF1 per mouse did not result in the activation of Akt.

The inventors also determined the kinetics with which Akt was activated following injection of mice with 5 μg IGF1. Salivary glands were removed at 5, 10, and 30 minutes post-injection, tissue lysates prepared, and the activation of Akt examined by immunoblotting with anti-phospho-Akt (threonine⁴⁷³) antibody (FIG. 12B). Maximal activation of Akt was detected five minutes after injection of IGF1, and the amount of phosphorylated Akt declined after this time; however, it could still be detected thirty minutes following administration of IGF1. Future studies are needed to determine whether Akt remains activated for longer periods of time.

The inventors also evaluated salivary flow rates three days after exposure to γ-irradiation. Injection of mice with 5 μg IGF1 alone had no effect upon the salivary flow rate three days following injection of the mice (FIG. 12C). There was a 40% decrease in the salivary flow rate in mice that received 1 Gy of irradiation compared to unirradiated control mice (FIG. 12C). Injection of mice with a single dose of 5 μg recombinant IGF1 completely suppressed the irradiation-induced reduction in salivary flow rate (FIG. 12C).

One hallmark of irradiation-induced salivary gland dysfunction is that the effect is chronic and there is no recovery in the production of saliva over time (Coppes et al., 2001). Therefore, the inventors quantitated salivary flow rates in irradiated FVB control, myr-Akt1 transgenic and IGF1 pretreated mice 30 days post γ-irradiation to determine if the trends observed three days following irradiation would be retained over time (FIG. 13). There was a slight increase in salivary flow rates in all unirradiated at day 30 when compared to mice when compared to the flow rates determined in unirradiated mice on day 3. The flow rate in the myr-Akt1 transgenic mice was higher than the unirradiated FVB control mice at the thirty day time point, and this difference was significant. There was not a statistical difference between unirradiated FVB control mice and mice injected with IGF1 at the thirty-day time point. There was a 60% decrease in the salivary flow rate in irradiated FVB mice compared to control mice at the thirty day time point. Although not statistically different, a 16% decrease in salivary flow was detected in myr-Akt1 mice exposed to γ-irradiation when compared to FVB untreated controls. Remarkably, FVB mice injected with a single dose of IGF1 prior to γ-irradiation treatment demonstrated no decrease in salivary flow rate 30 days after exposure compared to controls.

These data indicate that irradiation of FVB mice results in a significant induction of apoptosis and salivary gland dysfunction characterized by a decreased salivary flow rate that persists for at least 30 days. Expression of activated myr-Akt1, or pretreatment of mice with IGF1 which activates Akt, significantly decreases the number of apoptotic cells, and significantly suppresses γ-irradiation induced salivary gland dysfunction. It is logical to conclude that irradiation-induced apoptosis is causally related to salivary gland dysfunction. Furthermore, these results indicate that means to suppress apoptosis in salivary acinar cells could prevent inadvertent salivary gland dysfunction occurring secondary to head and neck irradiation. Current efforts are directed towards a preclinical model to evaluate the effectiveness of this approach.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference:
Anderson et al., Cell Death. Differ., 6:454-462, 1999.
Ashcroft et al., Mol. Cell. Biol., 19:1751-1758, 1999.
Attardi et al., Genes Dev., 14:704-718, 2000.
Bakkenist and Kastan, Nature, 421:499-506, 2003.
Bao et al., Nature, 411:969-974, 2001.
Bartek et al, Nat. Rev. Mol. Cell. Biol., 5(10):792-804, 2004.
Basu et al., Mol. Cell, 11:11-23, 2003.
Blattner et al., Oncogene, 18:1723-1732, 1999.
Bouvard et al., Oncogene, 19:649-660, 2000.
Brugarolas et al., Nature, 377:552-557, 1995.
Brunet et al., Cell, 96:857-868, 1999.
Cardone et al., Science, 282:1318-1321, 1998.
Chao et al., J. Biol. Chem., 278:41028-41033, 2003.
Chin et al., Oncogene, 15:87-99, 1997.
Coffer et al., Biochem. J., 335(1):1-13, 1998.
Coppes et al., Br. J. Cancer, 85:1055-1063, 2001
Datta et al., Cell, 91:231-241, 1997.
Datta et al., Genes Dev., 13:2905-2927, 1999.
Datta et al., J. Biol. Chem., 271:30835-30839, 1996.
Datta et al., Mol. Cell, 6:41-51, 2000.
del Peso et al., Oncogene, 18:7328-7333, 1999.
del Peso et al., Science, 278:687-689, 1997.
DeRyckere and DeGregori, Methods, 26:57-75, 2002.
Dietz et al., FEBS Lett., 525:93-99, 2002.
Dudek et al., Science, 275:661-665, 1997.
Feng et al., J. Biol. Chem., 279:35510-35517, 2004.
Flores et al., Cancer Cell, 7:363-373, 2005.
Flores et al., Nature, 416:560-564, 2002.
Fox et al., J. Am. Dent. Assoc., 110:519-525, 1985.
Gibson et al., J. Biol. Chem., 274:17612-17618, 1999.
Hamilton et al, J. Biol. Chem., 276:9028-9037, 2001.
Henrard and Ross, J. Virol., 62:3046-3049, 1988.
Henry et al., Blood, 98:834-841, 2001.
Humphries et al., J. Biol. Chem. 281:9728-9737 (2006).
Hutchinson et al., Mol. Cell. Biol., 21:2203-2212, 2001.
Inoki et al., Nat. Cell Biol., 4(9):648-657, 2002.
Jensen et al., J. Biol. Chem., 271:18749-18758, 1996.
Jonathan et al., Curr. Opin. Chem. Biol., 3:77-83, 1999.
Kandel and Hay, Exp. Cell Res., 253:210-229, 1999.
Kennedy et al., Mol. Cell. Biol., 19:5800-5810, 1999.
King et al., Cell Cycle, 3:634-637, 2004.
Kitagawa et al., FEBS Lett., 283:210-214, 1991.
Kulik et al., Mol. Cell. Biol., 17:1595-1606, 1997.
Lane and Lain, Trends Mol. Med., 8:S38-S42, 2002.
Lee et al., Horm. Metab. Res., 35:651-655, 2003.
Li et al., Science, 302:1972-1975, 2003.
Liem et al., Eur. J. Nucl. Med., 23:1485-1490, 1996.
Limesand et al., Cell Death. Differ., 10:345-355, 2003a.
Limesand et al., In Vitro Cell Dev. Biol. Anim, 39:170-177, 2003b.
Lin et al., Arch. Oral Biol., 46:1085-1089, 2001.
Maity et al., Radiother. Oncol., 31:1-13, 1994.
Matassa et al., J. Biol. Chem., 276:29719-29728, 2001.
Mayo and Donner, Proc. Natl. Acad. Sci. USA, 98:11598-11603, 2001.
Meek, Oncogene, 18:7666-7675, 1999.
Michael and Oren, Semin. Cancer Biol. 13:49-58, 2003.
Mitsiades et al., Oncogene, 21:5673-5683, 2002.
Miyashita et al., Oncogene, 9:1799-1805, 1994.
Momand et al., Gene, 242:15-29, 2000.
Nagler et al., Arch. Oral Biol., 43:297-303, 1998.
Nagler, In Vivo, 17:369-375, 2003.
Oda et al., Science, 288:1053-1058, 2000.
Ogawara et al., J. Biol. Chem., 277:21843-21850, 2002.
Okumura et al., Nat. Cell Biol., 4:111-116, 2002.
Oren et al., Ann. NY Acad. Sci., 973:374-383, 2002.
Oren, J. Biol. Chem., 274:36031-36034, 1999.
Paardekooper et al., Int. J. Radiat. Biol. 73:641-648, 1998.
Perry, Mol. Cancer Res., 2:9-19, 2004.
Potter et al., Nat. Cell Biol., 4(9):658-665, 2002.
Puc et al., Cancer Cell, 7:193-204, 2005.
Quissell et al., In Vitro Cell Dev. Biol. Anim, 33:164-173, 1997.
Remington's Pharmaceutical Sciences,” 15th Edition, chapter 33, in particular pages 624-652
Rudolph et al., J. Mammary Gland. Biol. Neoplasia., 8:287-307, 2003.
Sayan et al., Biochem. Biophys. Res. Commun., 330:186-193, 2005.
Schwertfeger et al., J. Lipid Res., 44:1100-1112, 2003.
Schwertfeger et al., Mol. Endocrinol., 15:867-881, 2001.
Senoo et al., Cancer Cell, 6:85-89, 2004.
Shioi et al., Mol. Cell Biol., 22:2799-2809, 2002.
Shioi et al., Mol. Cell Biol., 22:2799-2809, 2003.
Sluss et al., Mol. Cell Biol., 24:976-984, 2004.
Strano et al., J. Biol. Chem., 276:15164-15173, 2001.
Tibbetts et al., Genes Dev., 13:152-157, 1999.
Tuttle et al., Nat. Med., 7:1133-1137, 2001.
van Maanen et al., J. Natl. Cancer Inst., 80:1526-1533, 1988.
Vassilev et al., Science, 303:844-848, 2004.
Verheij and Bartelink, Cell Tissue Res., 301:133-142, 2000.
Viniegra et al., J. Biol. Chem., 280:4029-4036, 2005.
Vissink et al., Radiat. Res., 124:259-265, 1990.
Vogelstein et al., Nature, 408:307-310, 2000.
Wan and DeGregori, Immunity, 18:331-342, 2003.
Wang et al., FEBS Lett., 490:202-208, 2001.
Wendel et al., Nature, 428:332-337, 2004.
Woods and Vousden, Exp. Cell Res., 264:56-66, 2001.
Yang and McKeon, Nat. Rev. Mol. Cell Biol., 1:199-207, 2000.
Yin et al., Nat. Cell Biol., 4:462-467, 2002.
Yu et al., Mol. Cell, 7:673-682, 2001.
Zakowski and Bruns, Crit. Rev. Clin. Lab Sci., 21:283-322, 1985.
Zhou et al., Nat. Cell Biol., 3:973-982, 2001.

1. A method of reducing chemotherapy- or radiation-induced xerostomia in a subject having head & neck cancer comprising administering to said subject an inhibitor of apoptosis.

2. The method of claim 1, wherein said inhibitor is administered local to cancer.

3. The method of claim 2, wherein local administration is by injection into a salivary duct or oral lavage.

4. The method of claim 1, wherein said inhibitor is administered systemically.

5. The method of claim 1, wherein said inhibitor is an activator of the Akt pathway.

6. The method of claim 5, wherein said activator is IGF-1.

7. The method of claim 1, wherein said inhibitor is an inhibitor of the p53 pathway.

8. The method of claim 1, wherein said inhibitor is an activator of mdm2.

9. The method of claim 1, wherein said inhibitor is administered to said subject prior to receiving radiation or chemotherapy.

10. The method of claim 1, wherein said inhibitor is administered to said subject concurrent with receiving radiation or chemotherapy.

11. The method of claim 1, wherein said inhibitor is administered to said subject prior to and concurrent with receiving radiation or chemotherapy.

12. The method of claim 1, wherein said inhibitor is administered to said subject with 1 hour of receiving radiation or chemotherapy.

13. The method of claim 1, wherein said inhibitor is administered more than once.

14. The method of claim 1, further comprising administering chemotherapy or radiation therapy.

15. The method of claim 15, wherein radiation therapy comprises x-radiation, γ-radiation, or microwave radiation.

16. The method of claim 15, wherein chemotherapy comprises cisplatin, doxorubicin, taxol, etoposide, verapamil, podophyllotoxin, 5-fluorouracil (5-FU), cetuximab, docetaxel, carboplatin, and paclitaxel.

17. The method of claim 1, wherein said head & neck cancer is squamous cell carcinoma or adenocarcinoma.


SEQUENCE LISTING (10 sequences)

SEQ ID NO: 1 - DNA, 21 bp, Artificial Sequence
  Description of Artificial Sequence  Synthetic Primer
gccgctactatgccatgaaga

SEQ ID NO: 2 - DNA, 24 bp, Artificial Sequence
  Description of Artificial Sequence  Synthetic Primer
gtaatctggaacatcgtatgggta

SEQ ID NO: 3 - DNA, 21 bp, Artificial Sequence
  Description of Artificial Sequence  Synthetic Primer
atcattctgcccgagatggtg

SEQ ID NO: 4 - DNA, 21 bp, Artificial Sequence
  Description of Artificial Sequence  Synthetic Primer
tgctttacgggcttgtaggtg

SEQ ID NO: 5 - DNA, 18 bp, Artificial Sequence
  Description of Artificial Sequence  Synthetic Primer
gccacagcgaccatgtcc

SEQ ID NO: 6 - DNA, 22 bp, Artificial Sequence
  Description of Artificial Sequence  Synthetic Primer
gcgtctccgtgacgaagtcaaa

SEQ ID NO: 7 - DNA, 17 bp, Artificial Sequence
  Description of Artificial Sequence  Synthetic Primer
tcttcctcctccacctt

SEQ ID NO: 8 - DNA, 20 bp, Artificial Sequence
  Description of Artificial Sequence  Synthetic Primer
tgctgagcaaattgaactgc

SEQ ID NO: 9 - DNA, 20 bp, Artificial Sequence
  Description of Artificial Sequence  Synthetic Primer
cagcaccagcacctacttca

SEQ ID NO: 10 - DNA, 20 bp, Artificial Sequence
  Description of Artificial Sequence  Synthetic Primer
gataagctggctcacggaag